SEQUENCE LISTING (24 sequences)

SEQ ID NO: 1 - DNA, 360 bp, Artificial Sequence
  Heavy chain variable
gaagttcaacttgttgagagtggaggtggcttagttcaacctggtggatctcttagactctcttgtgctgcaagtggattcaatatcaaagatacttacattcattgggtgagacaagcacctggcaagggactagaatgggttgctaggatatacccaactaatggctatactagatatgctgatagtgtcaagggtagattcacaatttctgctgatacatcaaaaaacactgcttacttgcagatgaatagccttagagctgaggatacagcagtctactattgctcaagatggggtggggatggcttctatgctatggactattggggtcaaggaacattggtgacagtatcaagt

SEQ ID NO: 2 - protein, 120 aa, Artificial Sequence [sequence omitted]

SEQ ID NO: 3 - DNA, 324 bp, Artificial Sequence
  Light chain variable
gacattcaaatgactcaatccccatcaagtcttagcgcaagtgtgggagatcgtgtcactattacatgtcgagcatctcaagatgtgaatactgctgttgcgtggtaccaacaaaagcctggtaaagctccaaagttacttatatacagtgcaagctttctttatagtggcgtaccatctcgattcagtggatctcgaagtggaactgacttcaccttgactatctcatctctacaaccagaagacttcgctacttactattgtcaacaacattatacaactcctcctacattcgggcaaggtaccaaagttgagatcaagagg

SEQ ID NO: 4 - protein, 108 aa, Artificial Sequence
  Light chain variable
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 5 - DNA, 1347 bp, Artificial Sequence
  Heavy chain
gaagttcaacttgttgagagtggaggtggcttagttcaacctggtggatctcttagactctcttgtgctgcaagtggattcaatatcaaagatacttacattcattgggtgagacaagcacctggcaagggactagaatgggttgctaggatatacccaactaatggctatactagatatgctgatagtgtcaagggtagattcacaatttctgctgatacatcaaaaaacactgcttacttgcagatgaatagccttagagctgaggatacagcagtctactattgctcaagatggggtggggatggcttctatgctatggactattggggtcaaggaacattggtgacagtatcaagtgcttccaccaagggaccaagcgtttttcctttagccccaagttctaagtccactagtggaggtaccgcagctcttggttgtttagtcaaagattatttcccagagccagttaccgtgagttggaacagtggtgctttgactagtggagtccatacattcccagctgttttgcaatctagtggattgtattcactctctagtgtggttaccgtgccaagctcaagtttaggaacacaaacatatatatgcaatgtgaatcataaaccaagcaacactaaagttgataagaaagtggaaccaaagtcatgcgacaaaacacatacttgccctccatgccctgcacctgaattattgggaggtcctagtgtttttttatttccacctaaaccaaaagatacccttatgatttctaggacaccagaagttacttgtgtcgtggtcgatgtgtcccatgaagatccagaagttaaattcaattggtatgtggatggtgttgaagtgcataacgctaagactaagcctagggaggaacaatataattcaacttatagagtcgttagtgtccttactgtcctccaccaagattggttgaatggaaaggagtataaatgcaaagtctcaaataaggctctcccagcacctatcgaaaaaaccatatccaaggccaaaggacaacctagagagcctcaagtttatacacttccaccttctagggaagaaatgacaaagaaccaagtgagccttacatgtctcgttaagggtttctatcctagtgacattgccgttgaatgggagagtaatggacaacctgagaacaattataagactacacctccagtcttggatagtgatggttctttctttttgtattctaaattaactgttgacaaatcaagatggcaacagggaaatgttttttcatgttctgtcatgcacgaggctcttcacaatcattatactcaaaaatcacttagccttagcccagga

SEQ ID NO: 6 - protein, 449 aa, Artificial Sequence
  Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDTPPPCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - DNA, 642 bp, Artificial Sequence
  Light chain
gacattcaaatgactcaatccccatcaagtcttagcgcaagtgtgggagatcgtgtcactattacatgtcgagcatctcaagatgtgaatactgctgttgcgtggtaccaacaaaagcctggtaaagctccaaagttacttatatacagtgcaagctttctttatagtggcgtaccatctcgattcagtggatctcgaagtggaactgacttcaccttgactatctcatctctacaaccagaagacttcgctacttactattgtcaacaacattatacaactcctcctacattcgggcaaggtaccaaagttgagatcaagaggaccgtggctgcaccaagtgtgttcatatttcctccatccgatgaacaattgaagagtggtaccgcaagcgtcgtgtgtttattgaataacttttacccaagggaagccaaagttcaatggaaagttgataatgctctccaaagtggaaactcacaagaaagtgttacagagcaagactcaaaagattccacttatagcttatcaagtacacttactctctcaaaagcagactatgaaaaacacaaagtctacgcttgcgaagtcactcatcaaggactttcttcaccagttacaaagagtttcaatagaggagagtgt

SEQ ID NO: 8 - protein, 214 aa, Artificial Sequence
  Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1416 bp, Artificial Sequence
  Optimized heavy chain
atggctaaaacaaatctctttttattcttgattttctcccttttactttccttaagctcagcggaagttcaacttgttgagagtggaggtggcttagttcaacctggtggatctcttagactctcttgtgctgcaagtggattcaatatcaaagatacttacattcattgggtgagacaagcacctggcaagggactagaatgggttgctaggatatacccaactaatggctatactagatatgctgatagtgtcaagggtagattcacaatttctgctgatacatcaaaaaacactgcttacttgcagatgaatagccttagagctgaggatacagcagtctactattgctcaagatggggtggggatggcttctatgctatggactattggggtcaaggaacattggtgacagtatcaagtgcttccaccaagggaccaagcgtttttcctttagccccaagttctaagtccactagtggaggtaccgcagctcttggttgtttagtcaaagattatttcccagagccagttaccgtgagttggaacagtggtgctttgactagtggagtccatacattcccagctgttttgcaatctagtggattgtattcactctctagtgtggttaccgtgccaagctcaagtttaggaacacaaacatatatatgcaatgtgaatcataaaccaagcaacactaaagttgataagaaagtggaaccaaagtcatgcgacaaaacacatacttgccctccatgccctgcacctgaattattgggaggtcctagtgtttttttatttccacctaaaccaaaagatacccttatgatttctaggacaccagaagttacttgtgtcgtggtcgatgtgtcccatgaagatccagaagttaaattcaattggtatgtggatggtgttgaagtgcataacgctaagactaagcctagggaggaacaatataattcaacttatagagtcgttagtgtccttactgtcctccaccaagattggttgaatggaaaggagtataaatgcaaagtctcaaataaggctctcccagcacctatcgaaaaaaccatatccaaggccaaaggacaacctagagagcctcaagtttatacacttccaccttctagggaagaaatgacaaagaaccaagtgagccttacatgtctcgttaagggtttctatcctagtgacattgccgttgaatgggagagtaatggacaacctgagaacaattataagactacacctccagtcttggatagtgatggttctttctttttgtattctaaattaactgttgacaaatcaagatggcaacagggaaatgttttttcatgttctgtcatgcacgaggctcttcacaatcattatactcaaaaatcacttagccttagcccaggataatga

SEQ ID NO: 10 - protein, 470 aa, Artificial Sequence
  Optimized heavy chain
MAKTNLFLFLIFSLLLSLSSAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - DNA, 711 bp, Artificial Sequence
  Optimized light chain
atggctaaaacaaatctctttttattcttgattttctcccttttactttccttaagctcagcggacattcaaatgactcaatccccatcaagtcttagcgcaagtgtgggagatcgtgtcactattacatgtcgagcatctcaagatgtgaatactgctgttgcgtggtaccaacaaaagcctggtaaagctccaaagttacttatatacagtgcaagctttctttatagtggcgtaccatctcgattcagtggatctcgaagtggaactgacttcaccttgactatctcatctctacaaccagaagacttcgctacttactattgtcaacaacattatacaactcctcctacattcgggcaaggtaccaaagttgagatcaagaggaccgtggctgcaccaagtgtgttcatatttcctccatccgatgaacaattgaagagtggtaccgcaagcgtcgtgtgtttattgaataacttttacccaagggaagccaaagttcaatggaaagttgataatgctctccaaagtggaaactcacaagaaagtgttacagagcaagactcaaaagattccacttatagcttatcaagtacacttactctctcaaaagcagactatgaaaaacacaaagtctacgcttgcgaagtcactcatcaaggactttcttcaccagttacaaagagtttcaatagaggagagtgttaatga

SEQ ID NO: 12 - protein, 235 aa, Artificial Sequence
  Optimized light chain
MAKTNLFLFLIFSLLLSLSSADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 21 aa, Arabidopsis thaliana
MAKTNLFLFLIFSLLLSLSSA

SEQ ID NO: 14 - DNA, 10 bp, Artificial Sequence
  Poly A site
aatggaaatg

SEQ ID NO: 15 - DNA, 36 bp, Artificial Sequence
  Primer
gtgacagtatcaagtgcttccaccaagggaccaagc

SEQ ID NO: 16 - DNA, 36 bp, Artificial Sequence
  Primer
gcttggtcccttggtggaagcacttgatactgtcac

SEQ ID NO: 17 - DNA, 45 bp, Artificial Sequence
  Primer
cacttccaccttctagggaagaaatgacaaagaaccaagtgagcc

SEQ ID NO: 18 - DNA, 45 bp, Artificial Sequence
  Primer
ggctcacttggttctttgtcatttcttccctagaaggtggaagtg

SEQ ID NO: 19 - DNA, 99 bp, Artificial Sequence
  Primer
tttggtctcaaggtatggctaaaacaaatctctttttattcttgattttctcccttttactttccttaagctcagcggaagttcaacttgttgagagtg

SEQ ID NO: 20 - DNA, 38 bp, Artificial Sequence
  Primer
tttggtctcaaagctcattatcctgggctaaggctaag

SEQ ID NO: 21 - DNA, 36 bp, Artificial Sequence
  Primer
caaagttgagatcaagaggaccgtggctgcaccaag

SEQ ID NO: 22 - DNA, 36 bp, Artificial Sequence
  Primer
cttggtgcagccacggtcctcttgatctcaactttg

SEQ ID NO: 23 - DNA, 99 bp, Artificial Sequence
  Primer
tttggtctcaaggtatggctaaaacaaatctctttttattcttgattttctcccttttactttccttaagctcagcggacattcaaatgactcaatccc

SEQ ID NO: 24 - DNA, 37 bp, Artificial Sequence
  Primer
tttggtctcaaagctcattaacactctcctctattga

(19) United States
(12) Patent Application Publication
(10) Pub. No.: US 2012/0034207 A1
(43) Pub. Date: Feb. 9, 2012

(54) PRODUCTION OF HER RECEPTOR ANTIBODIES IN PLANT

(76) Inventors: MICHAEL D. MCLEAN, Guelph (CA); FREYDOUN GARABAGI, Guelph (CA); J. CHRISTOPHER HALL, Guelph (CA)

(21) Appl. No.: 13/161,987

(22) Filed: Jun. 16, 2011

Related U.S. Application Data
(60) Provisional application No. 61/355,300, filed on Jun. 16, 2010.

(51) Int. Cl. A61K 39/395; A61P 35/00; A01H 5/00; C12P 21/02; C07K 16/40

(52) U.S. Cl. 424/132.1; 435/69.6; 530/387.3; 800/298; 424/133.1

(57) ABSTRACT

A method of making an antibody in plants that binds to a HER receptor is described. The antibody preferably contains sequences from trastuzumab that have been optimized for expression in plants.

### RELATED APPLICATION

This application claims the benefit of U.S. provisional application 61/355,300 filed Jun. 16, 2010. The entire contents of which are herein incorporated by reference.

### INCORPORATION OF SEQUENCE LISTING

A computer readable form of the Sequence Listing “20436-16_SequenceListing.txt” (24,576 bytes), submitted via EFS-WEB and created on Jun. 16, 2011, is herein incorporated by reference.

### FIELD

The present application relates to methods of making an antibody or antibody fragment that binds to a human epidermal growth factor receptor (HER) receptor in a plant, the isolated antibodies or antibody fragments as well as methods of using same.

### BACKGROUND

Antibody research over the past 30 years has lead to the development of valuable biopharmaceuticals for the diagnosis and treatment of human disease (Nissim and Chernajovsky, 2008). To date, the United States Food and Drug Administration (FDA) has approved 22 monoclonal antibodies (mAb) for clinical use, while hundreds of others are in clinical trials (Chames et al. 2009; Dimitrov and Marks 2009). Antibodies currently approved for clinical therapy have a wide range of applications, including the treatment of microbial infections, autoimmune diseases and cancer (Chadd and Chamow, 2001; Stoger et al., 2005). The advantage of using antibodies in therapeutic applications is their low toxicity and high specificity for a target antigen (Ko et al., 2009); however, to ensure the efficiency of some treatments, high antibody serum concentrations must be maintained over a period of several months (Mori et al., 2007). One treatment cycle for a single patient can require hundreds of milligrams to gram quantities of mAbs (Leong and Chen, 2008; Mori et al., 2007). Therapeutic mAbs are thus among the most lucrative products within the biopharmaceutical industry (Karg and Kallio, 2009). From 2004 to 2006, market sales of the top five therapeutic mAbs (Rituxan®, Remicade®, Herceptin®, Humira®, and Avastin®) increased from $6.4 billion to $11.7 billion (Dimitrov et al., 2009). By 2010, the market value of these antibodies is predicted to rise to over $30 billion (Ko et al., 2009). In the past, such high market demands for biopharmaceuticals have lead to a manufacturing bottleneck (Karg et al., 2009).

Therapeutic mAbs have traditionally been produced in mammalian cell systems; however, high production costs and time-consuming culturing processes hinder the efficiency of these systems (Birch and Racher, 2006; Roque et al., 2007). In an attempt to meet rising market demands, pharmaceutical companies are working to improve the efficiency of existing biopharmaceutical production systems (Birch et al., 2006; Karg et al., 2009) as well as increase the number of antibody production facilities (Karg et al., 2009). Following construction, these facilities must be validated under Good Manufacturing Practice (GMP), a process that can take an average of three years (Vézina et al., 2009). Although some improvements have been made to increase antibody production, pharmaceutical companies still may not be able to meet future demands. As a result, alternative antibody expression systems are also being investigated (Birch et al., 2006; Karg et al., 2009).

Genetically modified plants offer an alternative to traditional mammalian cell expression systems for the large-scale production of therapeutic mAbs. In comparison to mammalian systems, genetically modified plants offer the advantages of lower upstream production costs, biological safety, and ease of handling. Conversely, the limitations of genetically modified plants include the addition of plant-specific N-glycans to the recombinant antibodies and high downstream processing and purification costs. A wide variety of transgenic plant hosts have been successfully used for recombinant antibody production. Tobacco has been one of the most important plants used for antibody expression as it has a large biomass and is not a food crop. Full-length recombinant antibodies were first successfully expressed in tobacco plants in 1989 (Hiatt et al., 1989). Since then, the expression of antibodies in tobacco has been achieved using different expression platforms, including both stable and transient plant transformation technologies (Ko et al., 2009; Giritch et al., 2006). Yet, despite the successful expression of antibodies in plants, there are currently no plant-produced antibodies that have been approved for human clinical therapy. To achieve regulatory affirmation of plant-produced therapeutics, researchers must be able to demonstrate that plant-produced antibodies maintain the identical structural and functional integrity as their mammalian counterparts (Stoger et al., 2005). Plant-produced antibody preparations must also be analyzed to ensure that they are homogeneous, nonimmunogenic and devoid of contaminants (Stoger et al., 2005). No study has been conducted to date to compare a plant-produced antibody with a clinically approved therapeutic mAb.

Trastuzumab (Herceptin® Genentech Inc., San Francisco, Calif.) is a humanized murine immunoglobulin G1κ antibody that is used in the treatment of metastatic breast cancer. Trastuzumab binds to the extracellular domain of human epidermal growth factor receptor 2 (HER2), a member of the ErbB family of transmembrane tyrosine kinase receptors, that is overexpressed in 20-30% of metastatic breast cancer patients (Ben-Kasus et al. 2009; Slamon et al. 1987, Slamon et al. 1989). Under normal cell conditions, HER2 is directly involved in the activation of signaling pathways that mediate normal cell growth and differentiation (Ben-Kasus et al., 2009; Hynes and Stern, 1994; Molina et al., 2001). Overexpression of HER2 results in the disruption of the normal signaling pathways, causing the loss of cell growth regulation and the development of resistance to apoptosis (Zhou et al., 2001; Le et al., 2003). By targeting cells that overexpress HER2, trastuzumab mediates the arrest of cell proliferation and the lysis of cancer cells by antibody-dependent cellular cytotoxicity (ADCC) (Arnould et al., 2006; Suzuki et al., 2007; Beano et al., 2008). In treatment, patients with HER2-overexpressing metastatic breast cancer are administered a loading dose of 4 mg of trastuzumab/kg followed by a weekly maintenance dose of 2 mg/kg (Cobleigh et al., 1999). Treatment of human metastatic breast cancer with trastuzumab thus requires kilogram quantities of this biopharmaceutical. In order to meet market demands, an efficient expression system is required for the large-scale production of trastuzumab.

### SUMMARY

The present inventors have successfully expressed trastuzumab in Nicotiana benthamiana using a viral-based transient expression system. Trastuzumab expression in N. benthamiana plants was quantified and plant-purified trastuzumab was characterized in comparison to commercial Herceptin. Plant-produced and commercial trastuzumab were found to have similar in vitro anti-proliferative effects on breast cancer cells that overexpress HER2. The results indicate that plant expression systems can effectively be used as an alternative to mammalian cell systems for large-scale production of therapeutic antibodies.

Accordingly, the present application provides a method of making an antibody or fragment thereof, that binds to a human epidermal growth factor receptor (HER) in a plant comprising:

(a) introducing a nucleic acid molecule encoding a heavy chain variable region and a nucleic acid molecule encoding a light chain variable region of the antibody into a plant or plant cell; and

(b) growing the plant or plant cell to obtain a plant that expresses the antibody or antibody fragment.

In one embodiment, the antibody or antibody fragment that has been prepared in a plant binds to the human epidermal growth factor receptor 2 (HER2).

In another embodiment, the nucleic acid molecules encoding the heavy chain (HC) and the light chain (LC) of trastuzumab and have been modified to incorporate plant preferred codons.

In a specific embodiment, the antibody or antibody fragment has the nucleic acid sequence of the heavy chain variable region as shown in SEQ ID NO:1 or the amino acid sequence of the heavy chain variable region shown in SEQ ID NO:2. In another embodiment, the antibody or antibody fragment has the nucleic acid sequence of the light chain variable region as shown in SEQ ID NO:3 or the amino acid sequence of the light chain variable region shown in SEQ ID NO:4.

The application further includes a transgenic plant that expresses an antibody that binds to a human epidermal growth factor receptor (HER) comprising a nucleic acid molecule encoding a heavy chain variable region and a nucleic acid encoding a light chain variable region of the antibody.

The application also includes methods of treating or diagnosing a cancer using the antibodies or antibody fragments of the application.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the schematic diagram of the T-DNA regions of pTrasHC (A) and pTrasLC (B). Both expression constructs contain the npt II gene under the control of the nopaline synthase promoter (NOSp). NOSt: nopaline synthase terminator; LB and RB: T-DNA left and right borders, respectively; AttB: recombination site; int: intron; SP: Arabidopsis basic chitinase signal peptide; HC: coding sequence for the heavy chain of trastuzumab; LC: coding sequence for the light chain of trastuzumab; 3′TMV: 3′ untranslated region of tobacco mosaic virus; 3′PVX: 3′ untranslated region of potato virus X.

FIG. 2 shows the quantification of trastuzumab expression in N. benthamiana. Trastuzumab was expressed in N. benthamiana using a viral-based transient expression system. Crude plant extracts were analyzed on a non-reducing immunoblot probed with anti-human IgG γ- and κ-chain specific probes. Lane 1: protein molecular weight standard; Lane 2-8: human IgG1, 1000, 500, 250, 125, 62.5, 31.3, 15.1 ng, respectively +10 μg total soluble protein (TSP) from untreated N. benthamiana; Lane 9: 10 μg TSP from untreated N. benthamiana; Lane 10-12: 10 μg TSP from three replicate N. benthamiana plants expressing trastuzumab. Molecular weights of protein standards are indicated on the left.

FIG. 3 shows the reducing Coomassie stained SDS-PAGE (A) and immunoblot (B) analyses of the purity of plant-produced trastuzumab. Lane 1: protein molecular weight standard; Lane 2: commercial Herceptin, 1.2 μg; Lane 3: plant-produced trastuzumab, 1.2 μg. Immunoblot was probed with anti-human IgG γ- and κ-chain specific probes. Molecular weights of protein standards are indicated on the left.

FIG. 4 shows the non-reducing immunoblot analyses of the purity and integrity of plant-produced trastuzumab, compared with human IgG1, commercial Herceptin and human serum IgG. Immunoblots were probed with (A) both anti-human IgG γ- and κ-chain specific probes, (B) γ-chain specific probe only, and (C) κ-chain specific probe only. Lane 1: protein standard; Lane 2: blank; Lane 3: human IgG1, 250 ng; Lane 4: commercial Herceptin, 250 ng; Lane 5: plant-produced trastuzumab, 250 ng; Lane 6: human serum IgG, 250 ng. Molecular weights of protein standards are indicated on the left.

FIG. 5 shows the qualitative analysis of the binding of plant-produced trastuzumab to HER2 ligand. MCF-7, BT-474 and SK-BR-3 cell lysates were analyzed by non-reducing immunoblots probed with (A) commercial Herceptin or (B) plant-produced trastuzumab. Lane 1: protein standard; Lane 2-4: 25 μg TSP of MCF-7, BT-474, and SK-BR-3 cell lysates, respectively.

FIG. 6 shows the effect of plant-produced trastuzumab on the proliferation of human breast tumor cells that overexpress HER2. BT-474 (A), SK-BR-3 (B) and MCF7 (C) cells were seeded into 6-well plates (5×10⁴ cells/well) and treated with no antibody, 2 μg/mL of non-specific plant-purified human IgG1 (negative control), 2 μg/mL of plant-produced trastuzumab or 2 μg/mL of commercial Herceptin. Cell counts were performed every two days to determine the relative cell proliferation. Data are expressed as a percentage of untreated control and are presented as means of triplicates±SEM.

### DETAILED DESCRIPTION

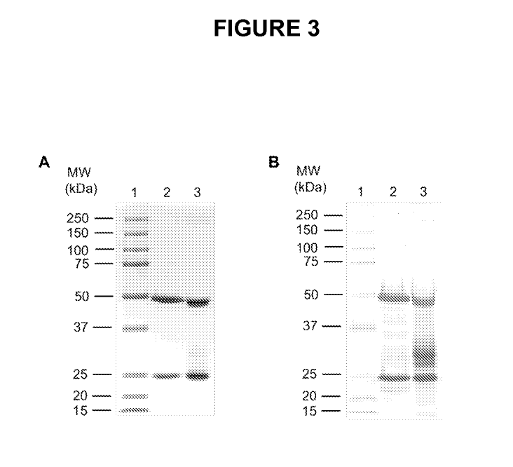

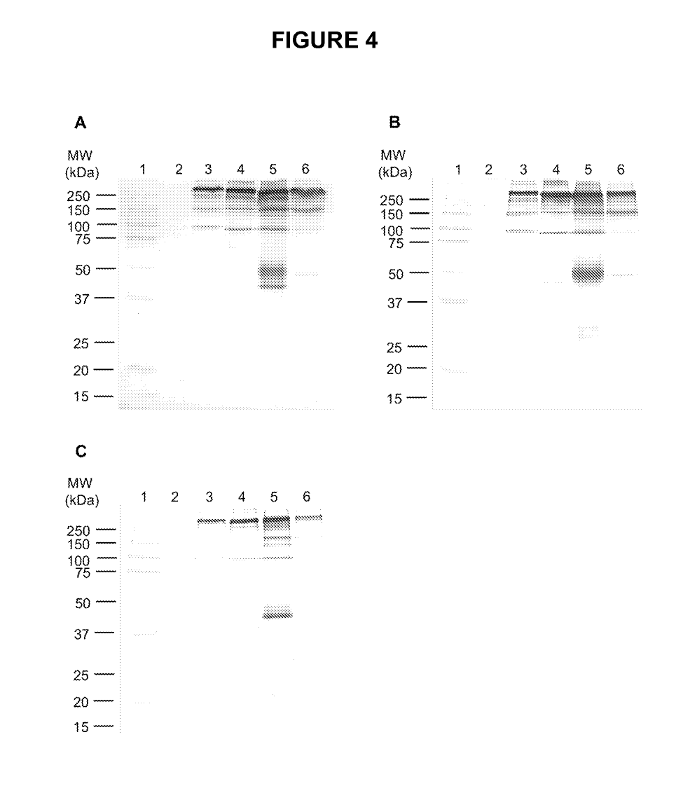

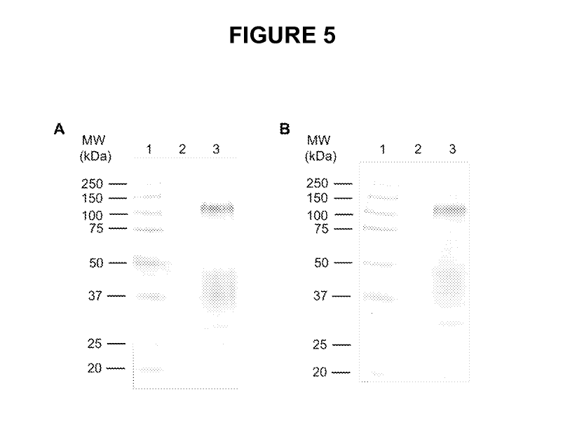

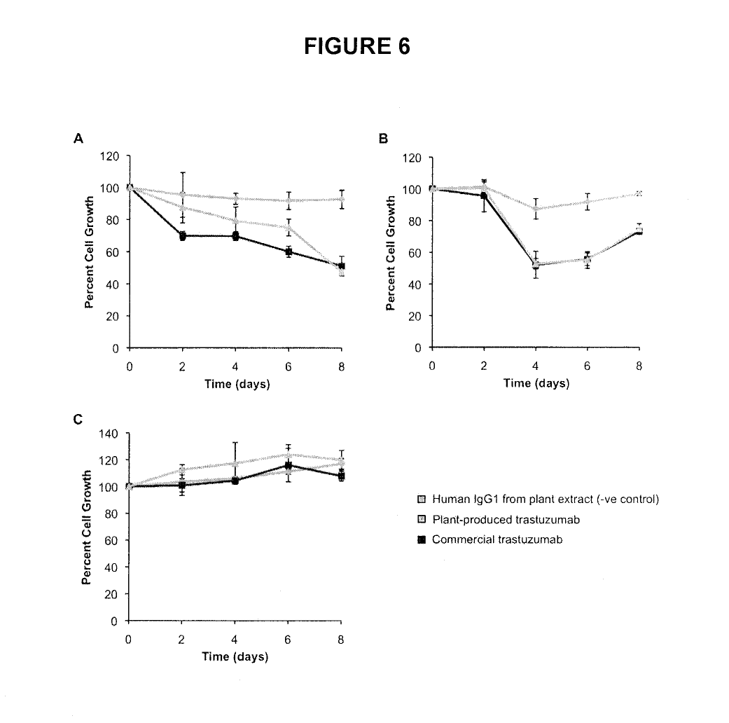

Trastuzumab (Herceptin®) was expressed in Nicotiana benthamiana plants using MagnICON® viral-based transient expression systems. Immunoblot analyses of crude plant extracts revealed that trastuzumab accumulates within plants mostly in the fully assembled tetrameric form (FIG. 2). Plants were determined to express 42 mg of trastuzumab per kilogram of fresh leaf tissue (0.6% TSP). Purification of trastuzumab from N. benthamiana tissue was achieved using a scheme that combines ammonium sulfate precipitation with affinity chromatography. Following purification, the specificity of the plant-produced trastuzumab for the HER2 receptor was compared with commercial Herceptin and confirmed by Western immunoblot (FIGS. 3 and 4). Functional assays revealed that plant-derived trastuzumab and commercial Herceptin both bind the HER2 protein in extracts of HER2 overexpressing cells (FIG. 5) and both have similar in vitro anti-proliferative effects on breast cancer cells that overexpress HER2 (FIG. 6). Results confirm that genetically modified plants can be used as an alternative to traditional antibody expression systems for the production of therapeutic mAbs.

Accordingly, the present invention provides a method of making an antibody or fragment thereof that binds to a HER receptor in a plant comprising:

(a) introducing a nucleic acid molecule encoding a heavy chain variable region and a light chain variable region of the antibody into a plant or plant cell; and

(b) growing the plant or plant cell to obtain a plant that expresses the antibody or antibody fragment.

In another embodiment, the application provides a method of making an antibody or fragment thereof that binds to a HER receptor in a plant comprising:

(a) introducing a nucleic acid molecule encoding a heavy chain and a nucleic acid molecule encoding a light chain of the antibody into a plant or plant cell; and

(b) growing the plant or plant cell to obtain a plant that expresses the antibody or antibody fragment.

As used herein, the term “antibody fragment” includes, without limitation, Fab, Fab′, F(ab′)₂, scFv, dsFv, ds-scFv, dimers, minibodies, diabodies, and multimers thereof and bispecific antibody fragments.

As used herein, the term “nucleic acid molecule” means a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

In one embodiment, the antibody or antibody fragment has the nucleic acid sequence of the heavy chain variable region as shown in SEQ ID NO:1 or the amino acid sequence of the heavy chain variable region shown in SEQ ID NO:2. In another embodiment, the antibody or antibody fragment has the nucleic acid sequence of the light chain variable region as shown in SEQ ID NO:3 or the amino acid sequence of the light chain variable region shown in SEQ ID NO:4.

In a specific embodiment, the antibody is trastuzumab or a modified form thereof, consisting of 2 HCs and 2 LCs. The heavy chain will preferably have the nucleic acid sequence shown in SEQ ID NO:5 or the amino acid sequence shown in SEQ ID NO:6. The light chain will preferably have the nucleic acid sequence shown in SEQ ID NO:7 or the amino acid sequence shown in SEQ ID NO:8.

In one embodiment, a signal peptide may be placed at the amino termini of the HC and/or LC. In a specific embodiment, the Arabidopsis thaliana basic chitinase signal peptide (SP), [(Samac et al., 1990)], namely MAKTNLFLFLIFSLLLSLSSA (SEQ ID NO:13), is placed at the amino-(N-) termini of both the HC and LC (Samac et al., 1990).

In a specific embodiment, the nucleic acid constructs are optimized for plant codon usage. In particular, the nucleic acid sequence encoding the heavy chain and light chain can be modified to incorporate preferred plant codons. In a specific embodiment, coding sequences for both the HC and LC, including the SP in both cases, were optimized for expression in Nicotiana species. The first goal of this procedure was to make the coding sequences more similar to those of Nicotiana species. Codon optimizations were performed utilizing online freeware, i.e., the Protein Back Translation program (Entelchon), and Nicotiana coding sequence preferences. Codons with the highest frequencies for each amino acid in Nicotiana species (Nakamura, 2005) were thereby incorporated. Furthermore, potential intervening sequence splice-site acceptor and donor motifs were identified (Shapiro et al., 1987; CNR National Research Council) and subsequently removed by replacement with nucleotides that resulted in codons encoding the same amino acids. Inverted repeat sequences were analyzed using the Genebee RNA Secondary Structure software package (Brodsky et al.; GeneBee Molecular Biology Server); nucleotides were changed to reduce the free energy (kilocalories per mole) of potential secondary structure while maintaining the polypeptide sequence. Likewise, repeated elements were analyzed (CNR National Research Council) and replaced where present. Potential methylation sites (i.e., CXG and CpG; Gardiner-Garden et al.) were replaced where possible and always without changing the encoded amino acid sequence. A Kozak (Kozak, 1984) optimized translation start site was engineered. Plant polyadenylation sites (i.e., AATAAA, AATGAA, AAATGGAAA, and AATGGAAATG (SEQ ID NO:14); Li et al.; Rothnie) and ATTTA RNA instability elements (Ohme-Takagi et al.) were likewise avoided.

The coding sequences for the HC and LC, including codons for the Arabidopsis basic chitinase SP, were synthesized using standard procedures (Almquist et al., Olea-Popelka et al. McLean et al). The entire SP-HC coding sequence was subcloned into pICH21595 (Giritch et al., 2006); Icon Genetics GmbH, Halle, Germany) to generate pTrasHC; the entire SP-LC coding sequence was subcloned into pICH25433 (Giritch et al., 2006) to generate pTrasLC.

The nucleic acid and amino acid sequence of the optimized heavy chain with the SP is shown in SEQ ID NOS:9 and 10, respectively. The nucleic acid and amino acid sequence of the optimized light chain with the SP is shown in SEQ ID NOS:11 and 12, respectively.

The nucleic acid constructs encoding the heavy chain variable region and/or the light chain variable region (and optionally the constant regions for both) will also contain other elements suitable for the proper expression of the antibodies or antibody fragments in the plant cell. In particular, each construct will also contain a promoter that promotes transcription in plant cells. Suitable promoters include, but are not limited to, cauliflower mosaic virus promoters (such as CaMV35S and 19S), nopaline synthase promoters; alfalfa mosaic virus promoter; other plant virus promoters. Constitutive promoters, such as plant actin gene promoters; histone gene promoters can also be used.

Inducible promoters, such as light-inducible promoters: ribulose-1,5-bisphosphate carboxylase oxidase (aka RUBISCO) small subunit gene promoter; chlorophyll a/b binding (CAB) protein gene promoter; and other light inducible promoters may also be used. Other inducible promoters include chemically-inducible promoters: alcohol inducible promoter; estrogen inducible promoter.

Synthetic promoters, such as the so-called superpromoter comprised of 3 mannopine synthase gene upstream activation sequences and the octopine synthase basal promoter sequence (Lee et al., 2007) can also be used.

Predicted promoters, such as can be found from genome database mining (Ilham et al., 2003) may also be used.

The nucleic acid constructs will also contain suitable terminators useful for terminating transcription in the plant cell. Examples of terminators include the nopaline synthase poly A addition sequence (nos poly A), cauliflower mosaic virus 19S terminator, actin gene terminator, alcohol dehydrogenase gene terminator, or any other terminator from the GenBank database.

The nucleic acid constructs may also include other components such as signal peptides that direct the polypeptide the secretory pathway of plant cells, such as the Arabidopsis thaliana basic chitinase signal peptide (SP), [(Samac et al., 1990)] as described above.

Other signal peptides can be mined from GenBank [http://www.ncbi.nlm.nih.gov/genbank/] or other such databases, and their sequences added to the N-termini of the HC or LC, nucleotides sequences for these being optimized for plant preferred codons as described above and then synthesized. The functionality of a SP sequence can be predicted using online freeware such as the SignalP program [http://www.cbs.dtu.dk/services/SignalP/].

Selectable marker genes can also be linked on the T-DNA, such as kanamycin resistance gene (also known as neomycin phonphatase gene II, or nptII), Basta resistance gene, hygromycin resistance gene, or others.

In another embodiment, the nucleic acid molecule encoding the heavy chain variable region and the nucleic acid molecule encoding the light chain variable region may be introduced into the plant cell on separate nucleic acid constructs. In such an embodiment, the heavy chain and the light chain would be expressed separately and then combine in the plant cell in order to prepare the antibody or antibody fragment that binds HER2.

In another embodiment, the nucleic acid molecule encoding the heavy chain variable region and the nucleic acid molecule encoding the light chain variable region may be introduced into the plant cell on the same nucleic acid construct.

The phrase “introducing a nucleic acid molecule into a plant or plant cell” includes both the stable integration of the nucleic acid molecule into the genome of a plant cell to prepare a transgenic plant as well as the transient integration of the nucleic acid into a plant or part thereof.

The nucleic acid constructs may be introduced into the plant cell using techniques known in the art including, without limitation, electroporation, an accelerated particle delivery method, a cell fusion method or by any other method to deliver the nucleic acid constructs to a plant cell, including Agrobacterium mediated delivery, or other bacterial delivery such as Rhizobium sp. NGR234, Sinorhizobium meliloti and Mesorhizobium loti (Chung et al, 2006].

The plant cell may be any plant cell, including, without limitation, tobacco plants, tomato plants, maize plants, alfalfa plants, Nicotiana benthamiana, rice plants, Lemna major or Lemna minor (duckweeds), safflower plants or any other plants that are both agriculturally propagated and amenable to genetic modification for the expression of recombinant or foreign proteins.

The phrase “growing a plant or plant cell to obtain a plant that expresses the antibody or antibody fragment” includes both growing transgenic plant cells into a mature plant as well as growing or culturing a mature plant that has received the nucleic acid molecules encoding the antibody. One of skill in the art can readily determine the appropriate growth conditions in each case.

In a specific embodiment, plasmids containing the nucleic acid molecules are introduced into A. tumefaciens strain by electroporation procedures. The N. benthamiana plants can be vacuum infiltrated according to the protocol described by Marillonnet et al. (2005) and Giritch et al. (2006) with several modifications. Briefly, all cultures can be grown at 28° C. and 220 rpm to a final optical density at 600 nm (OD₆₀₀) of 1.8. Equal volumes are combined and pelleted by centrifugation at 8,000 rpm for 4 minutes, resuspended and diluted by 10³ in infiltration buffer (10 mM 1-(N-morpholino)ethanesulphonic acid (MES) pH 5.5, 10 mM MgSO₄). Alternatively, each of the 5 Agrobacterium cultures could be grown to lower OD values and Beer's Law could be applied to determine the volumes of each culture required to make a bacterial suspension cocktail whereby the concentrations of each bacterial strain were equivalent. Alternatively, lower or higher concentrations of PVX vectors and TMV vectors could be used to optimize the expression of antibody. Alternatively, the SP-HC and SP-LC coding sequences could be subcloned into pICH25433 and pICH21595 respectively. Alternatively, higher or lower dilutions with infiltration buffer could be used.

The aerial parts of six-week-old N. benthamiana plants are submerged in a chamber containing the A. tumefaciens resuspension solution, after which a vacuum (0.5 to 0.9 bar) is applied for 90 seconds followed by a slow release of the vacuum, after which plants were returned to the greenhouse for 8 days before being harvested. Alternatively, longer or shorter periods under vacuum, and/or vacuum release, could either/or/and be used. Alternatively, longer or shorter periods of growth in greenhouse could be used. Alternatively, standard horticultural improvement of growth, maximized for recombinant protein production could be used (see Colgan et al., 2010).

Alternately, instead of transient introduction of TMV or PVX based vectors containing the trastuzumab HC and LC coding sequences, stable transgenic plants could be made using one binary vector on which the nucleic acid molecule encoding the heavy chain variable region and the nucleic acid molecule encoding the light chain variable region may be introduced together in the same construct. In one embodiment, the nucleic acid molecule encoding the heavy chain variable region may be attached to the nucleic acid molecule encoding the light chain variable region by a linker in order to prepare a single chain variable region fragment (scFv).

In another embodiment, the nucleic acid molecule encoding the heavy chain and the nucleic acid molecule encoding the light chain may be introduced into the plant cell on separate binary vector nucleic acid constructs. In such an embodiment, the heavy chain and the light chain would be expressed from separate transgenic loci and then combine in the plant cell in order to prepare the antibody or antibody fragment that binds HER2.

Binary plant expression vector(s) containing antibody HC and LC genes would be introduced into Agrobacterium tumefasciens At542 or other suitable Agrobacterium isolates or other suitable bacterial species capable of introducing DNA to plants for transformation such as Rhizobium sp., Sinorhizobium meliloti, Mesorhizobium loti and other species (Broothaerts et al. 2005; Chung et al., 2006), by electroporation or other bacterial transformation procedures. Agrobacterium clones containing binary vectors would be propagated on Luria-Bertani (LB) plates containing rifampicin (30 mg/l) and kanamycin (50 mg/l), or other selectable media, depending on the nature of the selectable marker genes on the binary vector. Agrobacterium-mediated leaf disk transformation (Horsch et al. 1985; Gelvin, 2003), or similar protocols involving wounded tobacco (N. tabacum, variety 81V9 or tissue of other tobacco varieties such as are listed in Conley et al, 2009) or N. benthamiana or other plant species such as those of the Solanaceae, maize, safflower, Lemna spp., etc. would be infected with the Agrobacterium culture (OD600=0.6) and plated on Murashige and Skoog plus vitamins medium (MS; Sigma), supplemented with agar (5.8%; Sigma) and containing kanamycin (100 mg/l) or 500 cefotaxime (mg/L) or other selectable media, depending on the nature of the selectable marker genes on the binary vector, for selection of transformed plant cells. Production of shoots would be induced with naphthalene acetic acid (NAA; 0.1 mg/l; Sigma) and benzyl adenine (BA; 1 mg/l; Sigma) in the medium. For induction of roots, the newly formed shoots were moved to Magenta boxes (Sigma-Aldrich, Oakville, ON) on MS medium (as above) that was lacking NAA and BA. After roots are formed, plants would be transplanted to soil and could be raised in greenhouse culture. For plant transformation, as many as possible or at least 25 primary transgenic plants would be produced. ELISA and quantitative immunoblots would be performed on each plant to characterize levels of total and active antibody produced by the plants, respectively (Almquist et al., 2004; 2006; McLean et al., 2007; Olea-Popelka et al., 2005; Makvandi-Nejad et al., 2005).

After selection of antibody expressing primary transgenic plants, or concurrent with selection of antibody expressing plants, derivation of homozygous stable transgenic plant lines would be performed. Primary transgenic plants would be grown to maturity, allowed to self-pollinate, and produce seed. Homozygosity would be verified by the observation of 100% resistance of seedlings on kanamycin plates (50 mg/L), or other selectable drug as indicated above. A homozygous line with single T-DNA insertions, that are shown by molecular analysis to produce most amounts of antibody, would be chosen for breeding to homozygosity and seed production, ensuring subsequent sources of seed for homogeneous production of antibody by the stable transgenic or genetically modified crop (Olea-Popelka et al., 2005; McLean et al., 2007; Yu et al., 2008).

Alternatively, the binary vector with both HC and LC genes, or 2 binary vectors (one with a HC gene and the other with a LC gene), could be used to transiently infect a plant or plant tissues, as described above, and tissue harvested as described above for subsequent purification of antibody.

The antibody or antibody fragment may be purified or isolated from the plants using techniques known in the art, including homogenization, clarification of homogenate and affinity purification. Homogenization is any process that crushes or breaks up plant tissues and cells and produces homogeneous liquids from plant tissues, such as using a blender, or juicer, or grinder, or pulverizer such as mortar and pestle, etc. Clarification involves either/and/or centrifugation, filtration, etc. Affinity purification uses Protein A or Protein G or Protein L or antibodies that bind antibodies.

The present application further includes a transgenic plant that expresses an antibody that binds to a human epidermal growth factor receptor (HER) comprising a nucleic acid molecule encoding a heavy chain variable region and a nucleic acid encoding a light chain variable region of the antibody.

The present application includes an antibody or antibody fragment prepared according to the method described herein. In one embodiment, the antibody comprises the heavy chain variable region shown in SEQ ID NO:2 and/or the light chain variable region shown in SEQ ID NO:4. In a specific embodiment, the antibody comprises the optimized heavy chain sequence shown in SEQ ID NO:10 and the optimized light chain sequence shown in SEQ ID NO:12.

The present application includes all uses of the antibodies prepared according to the method described herein, including, without limitation, the use in the diagnosis or therapy of cancers that overexpress HER2.

Accordingly, the present application provides a method of treating a cancer that overexpresses HER2 comprising administering an effective amount of an antibody or antibody fragment prepared in a plant as described herein.

The present application also provides a method of diagnosing a cancer that over expresses HER2 comprising:

(a) obtaining a sample from a patient suspected to have an HER2-associated cancer;

(b) contacting the sample with an antibody or antibody fragment described herein; and

(c) determining whether the antibody or antibody fragment binds to the sample wherein binding to the sample indicates that the sample is from a patient with an HER2-associated cancer.

The following non-limiting examples are illustrative of the present invention:

### Example 1

### Experimental Procedures

### Vector Construction and Plant Infiltration

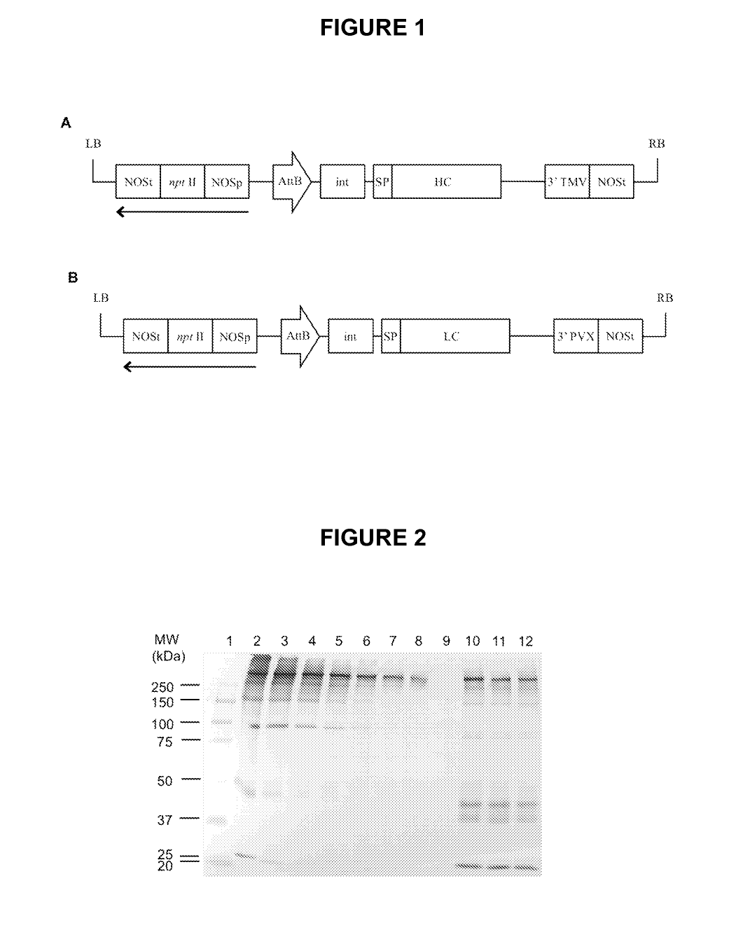

The variable coding regions of the heavy (V_{H}) and light (V_{L}) chains of trastuzumab (Carter et al., 1992) were synthesized as gene segments by the PBI/NRC DNA/Peptide Synthesis Laboratory of the National Research Council of Canada (Saskatoon, SK), incorporating preferred plant codons (Almquist et al., 2006; McLean et al., 2007; Olea-Popelka et al., 2005), a 24 amino acid N-terminal murine signal peptide (SP) (GenBank AAA38889), and 5′ XbaI and 3′ NotI restriction sites. The complete heavy chain coding sequence was assembled by subcloning murine SP-V_{H} into the XbaI/NotI sites of pMM3 (McLean et al., 2007), removing Lys₄₅₀, the NotI site, the six-Histidine and KDEL C-terminal tags, and changing Asp₃₅₉ to Glu and Leu₃₆₁ to Met by site directed mutagenesis. The complete heavy chain coding sequence including murine SP was amplified by PCR using primers containing BsaI sites and subcloned into pICH21595 (Icon Genetics GmbH, Munich, Germany) to generate pMTrasHC. The complete light chain coding sequence was assembled by sub-cloning murine SP-V_{L} into the XbaI/NotI sites of pMM7 (McLean et al., 2007). The NotI site was removed by site directed mutagenesis and the complete light chain coding sequence including murine SP was PCR amplified using primers containing BsaI sites and subcloned into pICH25433 (IconGenetics) to generate pMTrasLC. The Arabidopsis basic chitinase SP (Samac et al., 1990) later replaced the murine SP in both pMTrasHC and pMTrasLC, generating pTrasHC and pTrasLC, respectively (FIG. 1). All primers used for cloning of pTrasHC and pTrasLC are listed in Table 1 and 2, respectively.

The TMV-based 5′ module (pICH20111), PVX-based 5′ module (pICH24180) and integrase (pICH14011) vectors (IconGenetics) were unaltered. All five plasmids (pICH14011, pICH20111, pICH24180, pTrasHC and pTrasLC) were introduced into Agrobacterium tumefaciens strain At542 by electroporation. N. benthamiana plants were vacuum infiltrated according to the protocol described by Marillonnet et al. (2005) with several modifications. Briefly, all cultures were grown at 28° C. and 220 rpm to a final optical density at 600 nm (OD₆₀₀) of 1.8. Equal volumes were combined and pelleted by centrifugation at 8,000 rpm for 4 minutes, resuspended and diluted by 10⁻³ in infiltration buffer (10 mM 1-(N-morpholino)ethanesulphonic acid (MES) pH 5.5, 10 mM MgSO₄). The aerial parts of six-week-old N. benthamiana plants were submerged in a desiccator containing the A. tumefaciens resuspension under vacuum (0.5 to 0.9 bar) for 90 seconds before 60 second release, after which plants were returned to the greenhouse for 8 days before harvest.

### SDS-PAGE and Western Blot Analyses

Fresh leaf biomass from three N. benthamiana plants was harvested 8 d.p.i., ground separately under liquid nitrogen and combined with two volumes of cold extraction buffer [40 mM phosphate buffer pH 7.0, 50 mM ascorbic acid, 10 mM ethylenediaminetetraacetic acid disodium salt dihydrate (EDTA)]. Crude extracts were clarified by centrifugation at 10,000 rpm for 30 minutes and then 5,000 rpm for 10 minutes at 4° C. Total soluble protein (TSP) concentration was determined using the Bio-Rad Protein Assay (Mississauga, ON). Human myeloma IgG1 (Athens Research & Technology Inc, Athens, Ga.) was used as the protein standard. Western immunoblots were performed as described (Almquist et al., 2006), using a combination of goat anti-human IgG γ- and κ-chain specific probes conjugated to alkaline phosphatase (Sigma-Alderich, Oakville, ON), diluted to 1:2500 in phosphate-buffered saline (PBS), pH 7.6 containing 0.05% Tween-20.

### Quantitative ELISA

96-well microtiter plates (High-binding; Corning Inc Life Sciences, Lowell, Mass.) were coated overnight at 4° C. with 0.3125 μg/mL of mouse anti-human IgG γ-chain specific antibody (Sigma-Aldrich) diluted in PBS pH 7.4. Plates were blocked with 4% (w/v) skim milk (EMD Biosciences) dissolved in PBS for 24 hours at 4° C. and then washed five times with PBST. Serial dilutions of clarified extract from N. benthamiana plants expressing trastuzumab were added to plate, which was then incubated at 37° C. for 1 hour. Serial dilutions of human myeloma IgG1 (Athens Research & Technology Inc) normalized with clarified extract from untreated N. benthamiana plants were used as the standard. The plate was washed five times with PBST before adding polyclonal rabbit anti-human IgG (H+L)-horseradish peroxidase (HRP) conjugate (Abcam, Cambridge, AM), diluted to 1 μg/mL in PBS, for 1 hour at 37° C. The plate was washed five times with PBST before development with 1-Step™ Turbo TMB-ELISA (Thermo Scientific). Color development was stopped with 1.5 M sulfuric acid and optical densities were measured at 450 nm using an EnVision 2100 Multilabel microtitre plate reader (Perkin Elmer, Woodbridge, ON).

### Antibody Purification

Infiltrated N. benthamiana leaf tissue was harvested 8 d.p.i and stored at −80° C. Frozen leaf tissue (250 g) was combined with two volumes (500 mL) of cold extraction buffer in a food processor (Morphy Richards Inc, Mexborough, South Yorkshire, UK) and disrupted for three-30 seconds pulses. Disrupted tissue was collected and homogenized further using a benchtop polytron homogenizer (PT10/35, Kinematica Inc, Bohemia, N.Y.). Large plant debris was removed from the homogenate by dead-end filtration through miracloth (Calbiochem, San Diego, Calif.). Ammonium sulfate was slowly added to the filtered homogenate to a final concentration of 20%. The plant homogenate was then incubated at 4° C. for one hour with gentle stirring. Insoluble material was pelleted by centrifugation at 10,000 rpm for 30 minutes at 4° C. and the resulting supernatant collected. The concentration of ammonium sulfate in the resulting supernatant was subsequently increased to 60%, incubated at 4° C. for two hours with gentle stirring and centrifuged at 10,000 rpm for 30 minutes at 4° C. Pelleted protein was resuspended in 250 mL of 20 mM sodium phosphate, pH 7.0 and then passed through a series of filters (2.7 μm glass microfibre (GF/D), 1.2 μm glass microfibre (GF/C), 0.8 μm cellulose acetate, 0.45 μm cellulose acetate; Whatman, Piscataway, N.J.). The protein solution was dialysed and concentrated in a 250-mL Amicon ultrafiltration stirred cell (Millipore, Billerica, Mass.) with a molecular cutoff of 30 kDa (Millipore), then applied (4 mL/min) to a chromatography column (ID=2.5 cm; Bio-Rad) containing 10 mL of protein G Sepharose 4 Fast Flow affinity media (GE Healthcare, Baie d'Urfe, QC) pre-equilibrated with 20 mM phosphate buffer, pH 7.0. A series of washings were performed with 20 mM phosphate buffer, pH 7.0, to ensure the removal of all contaminating solutes from the protein G column. The antibody was eluted from the column with 0.1 M glycine pH 2.2 and immediately buffered with 1 M Tris.Cl pH 9.0. The buffered eluate was subsequently applied (2.5 mL/min) to a protein A affinity column (5 mL HiTrap™ Protein A HP column, GE Healthcare) connected to an AKTA-FPLC (Amersham Pharmacia Biotech, Uppsala, Sweden). To ensure the removal of all contaminating solutes from the protein A column, a series of washings were performed with 20 mM phosphate buffer, pH 7.0. The antibody was eluted from the column with 0.1 M glycine pH 2.2 and immediately buffered with 1 M Tris.Cl pH 9.0. Antibody eluate was dialysed against 20 mM phosphate buffer, pH 7.0 and concentrated using polyethylene glycol 35,000. Coomassie-stained SDS-PAGE gels and Western immunoblots were used to analyze the purity and structural integrity of plant-produced trastuzumab.

### N-Terminal Sequence Analysis

Plant-purified trastuzumab (3 μg) was separated by reducing 12% SDS-PAGE and then transferred to a Sequi-Blot™ PVDF membrane (Bio-Rad) which was treated with Coomassie blue R-250. N-terminal sequencing analysis (Edman degradation) was performed at the Hospital for Sick Children's Research Institute (The Advanced Protein Technology Centre, University of Toronto, Canada).

### Cell Culture

All mammary adenocarcinoma cell lines (MCF-7, SK-BR-3 and BT-474) were obtained from American Type Culture Collection (ATCC; Rockville, Md.) and cultured according to ATCC specifications for Western immunoblot analysis. Cell lysates were prepared from cell lines grown to 95% confluence and then treated with a 1× trypsin-EDTA solution (0.25% trypsin, 0.1% EDTA; SAFC Biosciences, Lenexa, Kans.), washed twice with ice-cold PBS, and treated with NP40 cell lysis buffer (50 mM Tris, pH 7.4, 250 mM NaCl, 5 mM EDTA, 50 mM NaF, 1 mM Na₃VO₄, 1% Nonidet P40, 0.02% NaN₃; Invitrogen) supplemented with 1 mM phenylmethanesulfonyl fluoride solution (PMSF; Sigma Aldrich) and 10% protease inhibitor cocktail (4-[2-aminoethyl]benzenesulfonyl fluoride, N-[trans-Epoxysuccinyl]-L-leucine 4-guanidinobutylamide, bestatin hydrochloride, leupeptin hemisulfate salt, aprotinin and sodium EDTA; Sigma-Aldrich). Total soluble protein (TSP) concentration was determined for each lysate using the BCA Protein Assay (Thermo Scientific). HER2 was detected in the cell lysate preparations using 0.1 μg/mL of either commercial Herceptin or plant-produced trastuzumab in PBST. Antibody samples were detected using a combination of goat anti-human IgG γ- and κ-chain specific probes conjugated to alkaline phosphatase (Sigma-Alderich), diluted to 1:2500 in PBST.

### Cell Proliferation Assay

MCF-7 and SK-BR-3 cell lines were cultured in Dulbecco's modified Eagle medium (DMEM) supplemented with 1 mg/mL fungizone, 1% penicillin/streptomycin (all from Invitrogen, Burlington, ON), and 10% fetal bovine serum (FBS; Sigma-Alderich). BT-474 cell line was cultured in Roswell Park Memorial Institute (RPMI) 1640 basal medium (Invitrogen) supplemented with 1 mg/mL fungizone, 1% penicillin/streptomycin, and 10% FBS. SK-BR-3, BT-474 and MCF7 cells were seeded into 6-well plates (Corning, Lowell, Mass.) (5×10⁴ cells/well). After allowing the cells to adhere, the cells were treated with no antibody, 2 μg/mL of non-specific plant-purified human IgG1 (negative control), 2 μg/mL of plant-produced trastuzumab, or 2 μg/mL of commercial Herceptin. Relative cell proliferation was determined by viable cell counts using trypan blue stain (Invitrogen). Cell counts were performed every two days for a total of eight days. Data are expressed as a percentage of untreated control.

### Results

Accumulation of Trastuzumab in N. benthamiana Plants

Trastuzumab was expressed in N. benthamiana plants using a viral-based transient expression system. Six-week old N. benthamiana plants were vacuum-infiltrated with A. tumefaciens clones transformed with provectors containing the HC- and LC-coding sequences of trastuzumab. Results of preliminary experiments determined that the murine SP did not allow much accumulation of trastuzumab (not shown); therefore, the murine SP was replaced by the Arabidopsis basic chitinase SP on both HC- and LC-expression constructs. The assembly of trastuzumab with the Arabidopsis SP-containing constructs was examined 8 days post infiltration (d.p.i) on a non-reducing Western immunoblot treated with a combination of anti-human IgG γ- and κ-chain specific probes. As shown in FIG. 2, the tetrameric form of the antibody (H₂L₂) was the most prominent band. Trastuzumab expression was also determined by non-reducing immunoblot and confirmed by quantitative ELISA through comparison with known concentrations of a human IgG1 standard (not shown). Plants were determined to express 60±7 mg of trastuzumab per kilogram of fresh leaf tissue (0.9±0.1% TSP).

### Purification and Characterization of Plant-Produced Trastuzumab

A scalable purification scheme was developed to facilitate the recovery of trastuzumab from N. benthamiana plants. Primary plant extracts were treated with 20% ammonium sulfate to remove high molecular weight contaminants, followed by 60% ammonium sulfate to enrich antibody yield through precipitation. Trastuzumab was subsequently purified by both protein G and then protein A affinity chromatography. Following purification, the plant-produced trastuzumab was analyzed and compared to commercial Herceptin by reducing SDS-PAGE stained with Coomassie blue. As seen in FIG. 3A, two major bands observed at approximately 50 kDa and 25 kDa are the heavy and light chains of trastuzumab, respectively. The heavy chain of plant-produced trastuzumab migrated slightly faster than the heavy chain of commercial Herceptin, likely due to differences between plant and mammalian post-translational glycosylation. There were no detectable differences in the electrophoretic mobilities of the light chains of commercial Herceptin and plant-produced trastuzumab. In addition to the bands representing the heavy and light chains of trastuzumab, two less prominent bands were observed between 25 and 37 kDa; these were enhanced by immunoblotting (FIG. 3B; see below for identification).

The structural integrity of plant-produced trastuzumab was analyzed on a non-reducing Western immunoblot treated with a combination of γ- and κ-chain specific probes. Plant-produced trastuzumab was also compared to human IgG1, commercial Herceptin and human serum IgG. All antibody samples contained bands with similar electrophoretic mobilities; however, plant-produced trastuzumab had four additional bands (marked by asterisks in FIG. 4A). Further examination of plant-produced trastuzumab on a Western immunoblot probed with a γ-chain specific probe revealed that the band at approximately 50 kDa represents unassembled heavy chains and the two bands between 25 and 37 kDa (described above) represent a heavy chain degradation product (FIG. 4B). Plant-produced trastuzumab was also examined on a Western immunoblot probed with a κ-chain specific probe. It appears that the bands at approximately 45 kDa and 25 kDa represent Fab fragments and unassembled light chains, respectively (FIG. 4C).

N-terminal sequencing by Edman degradation indicated 100% cleavage of the Arabidopsis SP from both the heavy- and light-chains of the plant-produced trastuzumab (not shown).

### Specificity of Plant-Produced Trastuzumab

A qualitative binding analysis was performed to demonstrate the specificity of plant-produced trastuzumab for HER2. MCF-7 and BT-474 cell lysates were resolved on a Western immunoblot that was subsequently probed with either plant-produced trastuzumab or commercial Herceptin. One band was observed on both of the immunoblots probed with either mAb (FIG. 5). The single band on both immunoblots corresponds to HER2 from the BT-474 cell lysates. No bands were observed in the lane containing the MCF-7 cells lysates, as this cell line does not overexpress HER2. The specificity of plant-produced trastuzumab for HER2 was also confirmed by qualitative ELISA (not shown).

### Inhibition of Tumor Cell Proliferation

The effect of plant-produced trastuzumab on the growth of breast tumor cells that overexpress HER2 was examined using a cell proliferation assay. Both HER2 overexpressing tumor cells (BT-474 and SK-BR-3) and normal HER2 expressing tumor cells (MCF7) were treated with commercial Herceptin or plant-produced trastuzumab. After 8 days, both plant-produced trastuzumab and commercial Herceptin showed 52.5% and 48.8% inhibition of BT-474 cell proliferation, respectively (FIG. 6A). After 4 days, plant-produced trastuzumab and commercial Herceptin showed 47.1% and 47.8% inhibition of SK-BR-3 cell proliferation, respectively (FIG. 6B). As shown in FIG. 6C, plant-produced trastuzumab and commercial Herceptin had no anti-proliferative effect on MCF7 cells. Plant-produced trastuzumab thus selectively inhibits the proliferation of both BT-474 and SK-BR-3 cells. As a negative control, all breast tumor cell lines were also treated with a non-specific plant-purified human IgG1. This non-specific plant-purified antibody had no effect on the breast tumor cell proliferation, which demonstrates the absence of plant contaminants that could inhibit the proliferation of breast tumor cells.

### Discussion

Genetically modified plants offer an alternative to traditional mammalian cell expression systems for the large-scale production of therapeutic mAbs. Yet, despite the successful expression of antibodies in plants, strict regulations pertaining to the safety and efficacy of plant-produced mAbs must be adhered to before approval for human therapy. Although one plant-produced mAb will soon enter human clinical trials (Gilbert, 2009; Ramessar et al., 2008), future validation of plant-produced therapeutic mAbs will require that they be shown to have similar biological properties (i.e., bioactivity and biosafety) to clinically approved parental mAbs.

Numerous researchers have shown that plant-produced mAbs retain biological activities (i.e., specificity, cytotoxicity and neutralization activity) that are similar to parental mAbs produced in mammalian cell culture (Table 3); however, no study has yet been conducted to characterize and compare a plant-produced mAb to a clinically approved therapeutic antibody with the identical primary structure. To date, TeraCIM®, an anti-epidermal growth factor receptor (EGF-R) antibody with conditioned registry approval in Cuba, is the only clinically approved mAb produced by plants (Rodríguez et al., 2005). Although it was determined that the plant-produced antibody and TeraCIM® have similar binding to A431 human tumor cell culture cells, the plant-produced antibody was modified to remove glycosylation sites and to add a KDEL ER-retention signal (Rodríguez et al., 2005).

The present application on the expression and purification of the anti-breast cancer antibody trastuzumab contributes further evidence that genetically modified plants can be used for the production of therapeutic mAbs, as the inventors were able to produce trastuzumab in N. benthamiana with identical primary structures to its heavy and light chains. Although our primary plant extracts revealed that N. benthamiana plants express 42±7 mg of trastuzumab per kilogram of fresh weight (0.6±0.1% TSP), optimization of this expression system should allow expression of up to 500 mg per kg FW (Marillonnet et al., 2005). Further, plant-produced trastuzumab was found to have similar specificity to commercial Herceptin for HER2, and was determined to be as effective as commercial Herceptin in inhibiting the growth of cells overexpressing HER2, while having no effect on cells with normal levels of HER2.

This example clearly shows that a plant-expression and purification system can produce a therapeutic mAb with identical primary structures and similar in vitro bioactivities to its parental mAb, supporting plant expression systems as effective alternatives to mammalian cell systems for the production of therapeutic mAbs.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Nucleotide sequences of the primers used in the construction of
pTrasHC.

| | | |
|---|---|---|
| Name | Type | Nucleotide Sequence |

Removal of NotI site

| | | |
|---|---|---|
| TrasHC-NotI | Forward | 5′-GTGACAGTATCAAGTGCTTCCACCAAGGGACCAAGC-3′ |
| | | (SEQ ID NO: 15) |
| | Reverse | 5′-GCTTGGTCCCTTGGTGGAAGCACTTGATACTGTCAC-3′ |
| | | (SEQ ID NO: 16) |

Amino acid modification (Asp₃₅₉ → Glu; Leu₃₆₁ → Met)

| | | |
|---|---|---|
| TrasHC-2AA | Forward | 5′-CACTTCCACCTTCTAGGGAAGAAATGACAAAGAACCAAGTG |
| | | AGCC-3′ |
| | | (SEQ ID NO: 17) |
| | Reverse | 5′-GGCTCACTTGGTTCTTTGTCATTTCTTCCCTAGAAGGTGGAA |
| | | GTG-3′ |
| | | (SEQ ID NO: 18) |

Subcloning into pICH21595 (addition of Arabidopsis basic
chitinase SP, removal of Lys₄₅₀, 6xHis and KDEL tags)

| | | |
|---|---|---|
| TrasHC-S | Forward | 5′-TTTGGTCTCAAGGTATGGCTAAAACAAATCTCTTTTTATTCTT |
| | | GATTTTCTCCCTTTTACTTTCCTTAAGCTCAGCGGAAGTTCAAC |
| | | T TGTTGAGAGTG-3′ |
| | | (SEQ ID NO: 19) |
| | Reverse | 5′-TTTGGTCTCAAAGCTCATTATCCTGGGCTAAGGCTAAG-3′ |
| | | (SEQ ID NO: 20) |

TABLE 2
Nucleotide sequences of the primers used in the construction of
pTrasLC.

| | | |
|---|---|---|
| Name | Type | Nucleotide Sequence |

Removal of NotI site

| | | |
|---|---|---|
| TrasLC-NotI | Forward | 5′-CAAAGTTGAGATCAAGAGGACCGTGGCTGCACCAAG-3′ |
| | | (SEQ ID NO: 21) |
| | Reverse | 5′-CTTGGTGCAGCCACGGTCCTCTTGATCTCAACTTTG-3′ |
| | | (SEQ ID NO: 22) |

Subcloning into pICH25433 (addition of Arabidopsis basic
chitinase SP)

| | | |
|---|---|---|
| TrasLC-S | Forward | 5′-TTTGGTCTCAAGGTATGGCTAAAACAAATCTCTTTTTATTCTT |
| | | GATTTTCTCCCTTTTACTTTCCTTAAGCTCAGCGGACATTCAAAT |
| | | GACTCAATCCC-3′ |
| | | (SEQ ID NO: 23) |
| | Reverse | 5′-TTTGGTCTCAAAGCTCATTAACACTCTCCTCTATTGA-3′ |
| | | (SEQ ID NO: 24) |

TABLE 3
Studies that compare plant-produced mAbs with their parental mammalian cell culture-produced mAbs (adapted from
Fischer et al. (2009)).

| | | | | Difference | | | |
|---|---|---|---|---|---|---|---|
| | | | | from | | | |
| | | | Expression | Parental | | | |
| Antibody | Antigen | Application | Host | mAb | Yield | Comments | References |
| 2G12 | HIV gp120 | Topical | South | Primary | 75 μg of Ab/g | Equivalent binding activity as mAb^{M} | (Ramessar et |
| Human | | application | African | structure | dry seed | Presence of heavy chain degradation products | al., 2008) |
| IgG1 | | | elite white | assumed | weight | that do not bind target | |
| | | | maize | identical^{†} | | 3X more efficient than ^{CHO}2G12 in HIV | |
| | | | (M37W) | | | neutralization assay | |
| 2F5 | HIV gp41 | Microbicide | Transgenic | KDEL tag | 1.8 mg/L | 85% of the binding capacity of ^{CHO}2F5 | Sack et al., |
| Human | | | tobacco BY2 | | suspension | 3X less efficient than ^{CHO}2F5 in HIV | 2007 |
| IgG1 | | | suspension | | culture | neutralization assay | |
| | | | cell cultures | | | | |
| 2F5 | HIV gp41 | Microbicide | N. tabacum | KDEL tag | 0.1-0.6% TSP | Same binding kinetics as ^{CHO}2F5 | Floss et al., |
| Human | | | | | | | 2008 |
| IgG1 | | | | | | | |
| BR55-2 | Lewis Y | Anti-breast | N. tabacum | KDEL tag | 31 mg/kg of | Comparison of mAb^{P} and mAb^{M} revealed | Brodzik et al., |
| Murine | oligosaccharide | and- | (LAMD609) | | fresh leaf | similar: | 2006 |
| IgG2a | | colorectal | | | tissue | specificity for SK-BR-3 and SW948 cells | |
| | | cancer | | | | binding to FcγRI receptor (CD64) | |
| | | antibody | | | | in vitro cytotoxicity against SK-BR3 cells | |
| | | | | | | in vivo tumor inhibition | |
| CO17-1A | Tumor- | Anti- | N. tabacum | Primary | 0.9 mg/kg of | Similar binding activity as ^{CHO}CO17-1A | Ko et al., 2005 |
| Murine | associated | colorectal | (Xanthi) | structure | fresh leaf | Able to suppress human colorectal tumor | |
| IgG2a | antigen | cancer Ab | | assumed | tissue | growth as effectively as ^{CHO}CO17-1A | |
| | GA733-2 | | | identical^{†} | 0.02% TSP | mAbP and mAbM have similar binding activity | Jamal et al., |
| | | | | | | to Fc receptor FcγRI (CD64) | 2009 |
| | | | | | | Concluded that altered glycosylation pattern | |
| | | | | | | does not affect binding to CD64 | |

SEQUENCE LISTING
SEQ ID NO: 1
nucleic acid sequence of the heavy chain
variable region of trastuzumab
GAAGTTCAACTTGTTGAGAGTGGAGGTGGCTTAGTTCAACCTGGTGGATC
TCTTAGACTCTCTTGTGCTGCAAGTGGATTCAATATCAAAGATACTTACA
TTCATTGGGTGAGACAAGCACCTGGCAAGGGACTAGAATGGGTTGCTAGG
ATATACCCAACTAATGGCTATACTAGATATGCTGATAGTGTCAAGGGTAG
ATTCACAATTTCTGCTGATACATCAAAAAACACTGCTTACTTGCAGATGA
ATAGCCTTAGAGCTGAGGATACAGCAGTCTACTATTGCTCAAGATGGGGT
GGGGATGGCTTCTATGCTATGGACTATTGGGGTCAAGGAACATTGGTGAC
AGTATCAAGT
SEQ ID NO: 2
amino acid sequence of the heavy chain
variable region of trastuzumab
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
SEQ ID NO: 3
nucleic acid sequence of the light chain
variable region of trastuzumab
GACATTCAAATGACTCAATCCCCATCAAGTCTTAGCGCAAGTGTGGGAGA
TCGTGTCACTATTACATGTCGAGCATCTCAAGATGTGAATACTGCTGTTG
CGTGGTACCAACAAAAGCCTGGTAAAGCTCCAAAGTTACTTATATACAGT
GCAAGCTTTCTTTATAGTGGCGTACCATCTCGATTCAGTGGATCTCGAAG
TGGAACTGACTTCACCTTGACTATCTCATCTCTACAACCAGAAGACTTCG
CTACTTACTATTGTCAACAACATTATACAACTCCTCCTACATTCGGGCAA
GGTACCAAAGTTGAGATCAAGAGG
SEQ ID NO: 4
amino acid sequence of the light chain
variable region of trastuzumab
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKR
SEQ ID NO: 5
nucleic acid coding sequence of the heavy
chain of trastuzumab
GAAGTTCAACTTGTTGAGAGTGGAGGTGGCTTAGTTCAACCTGGTGGATC
TCTTAGACTCTCTTGTGCTGCAAGTGGATTCAATATCAAAGATACTTACA
TTCATTGGGTGAGACAAGCACCTGGCAAGGGACTAGAATGGGTTGCTAGG
ATATACCCAACTAATGGCTATACTAGATATGCTGATAGTGTCAAGGGTAG
ATTCACAATTTCTGCTGATACATCAAAAAACACTGCTTACTTGCAGATGA
ATAGCCTTAGAGCTGAGGATACAGCAGTCTACTATTGCTCAAGATGGGGT
GGGGATGGCTTCTATGCTATGGACTATTGGGGTCAAGGAACATTGGTGAC
AGTATCAAGTGCTTCCACCAAGGGACCAAGCGTTTTTCCTTTAGCCCCAA
GTTCTAAGTCCACTAGTGGAGGTACCGCAGCTCTTGGTTGTTTAGTCAAA
GATTATTTCCCAGAGCCAGTTACCGTGAGTTGGAACAGTGGTGCTTTGAC
TAGTGGAGTCCATACATTCCCAGCTGTTTTGCAATCTAGTGGATTGTATT
CACTCTCTAGTGTGGTTACCGTGCCAAGCTCAAGTTTAGGAACACAAACA
TATATATGCAATGTGAATCATAAACCAAGCAACACTAAAGTTGATAAGAA
AGTGGAACCAAAGTCATGCGACAAAACACATACTTGCCCTCCATGCCCTG
CACCTGAATTATTGGGAGGTCCTAGTGTTTTTTTATTTCCACCTAAACCA
AAAGATACCCTTATGATTTCTAGGACACCAGAAGTTACTTGTGTCGTGGT
CGATGTGTCCCATGAAGATCCAGAAGTTAAATTCAATTGGTATGTGGATG
GTGTTGAAGTGCATAACGCTAAGACTAAGCCTAGGGAGGAACAATATAAT
TCAACTTATAGAGTCGTTAGTGTCCTTACTGTCCTCCACCAAGATTGGTT
GAATGGAAAGGAGTATAAATGCAAAGTCTCAAATAAGGCTCTCCCAGCAC
CTATCGAAAAAACCATATCCAAGGCCAAAGGACAACCTAGAGAGCCTCAA
GTTTATACACTTCCACCTTCTAGGGAAGAAATGACAAAGAACCAAGTGAG
CCTTACATGTCTCGTTAAGGGTTTCTATCCTAGTGACATTGCCGTTGAAT
GGGAGAGTAATGGACAACCTGAGAACAATTATAAGACTACACCTCCAGTC
TTGGATAGTGATGGTTCTTTCTTTTTGTATTCTAAATTAACTGTTGACAA
ATCAAGATGGCAACAGGGAAATGTTTTTTCATGTTCTGTCATGCACGAGG
CTCTTCACAATCATTATACTCAAAAATCACTTAGCCTTAGCCCAGGA
SEQ ID NO: 6
Amino acid sequence of the heavy chain of
trastuzumab (i.e., H-GAMMA-1(V_{H}: 1-120 + CH1:
121-218) + HINGE-REGION(219-233) + C_{H2}:
234-343) + C_{H3}: 344-450))
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDTPPPCPRCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
SEQ ID NO: 7
nucleic acid coding sequence of the light
chain of trastuzumab
GACATTCAAATGACTCAATCCCCATCAAGTCTTAGCGCAAGTGTGGGAGA
TCGTGTCACTATTACATGTCGAGCATCTCAAGATGTGAATACTGCTGTTG
CGTGGTACCAACAAAAGCCTGGTAAAGCTCCAAAGTTACTTATATACAGT
GCAAGCTTTCTTTATAGTGGCGTACCATCTCGATTCAGTGGATCTCGAAG
TGGAACTGACTTCACCTTGACTATCTCATCTCTACAACCAGAAGACTTCG
CTACTTACTATTGTCAACAACATTATACAACTCCTCCTACATTCGGGCAA
GGTACCAAAGTTGAGATCAAGAGGACCGTGGCTGCACCAAGTGTGTTCAT
ATTTCCTCCATCCGATGAACAATTGAAGAGTGGTACCGCAAGCGTCGTGT
GTTTATTGAATAACTTTTACCCAAGGGAAGCCAAAGTTCAATGGAAAGTT
GATAATGCTCTCCAAAGTGGAAACTCACAAGAAAGTGTTACAGAGCAAGA
CTCAAAAGATTCCACTTATAGCTTATCAAGTACACTTACTCTCTCAAAAG
CAGACTATGAAAAACACAAAGTCTACGCTTGCGAAGTCACTCATCAAGGA
CTTTCTTCACCAGTTACAAAGAGTTTCAATAGAGGAGAGTGT
NO: 8
amino acid sequence of the light chain of
trastuzumab (i.e., V-KAPPA (V_{L}: 1-107) +
C-KAPPA(CL: 108-214))
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 9
nucleic acid sequence of the optimized
heavy chain coding sequence of trastuzumab,
including signal peptide
ATGGCTAAAACAAATCTCTTTTTATTCTTGATTTTCTCCCTTTTACTTTC
CTTAAGCTCAGCGGAAGTTCAACTTGTTGAGAGTGGAGGTGGCTTAGTTC
AACCTGGTGGATCTCTTAGACTCTCTTGTGCTGCAAGTGGATTCAATATC
AAAGATACTTACATTCATTGGGTGAGACAAGCACCTGGCAAGGGACTAGA
ATGGGTTGCTAGGATATACCCAACTAATGGCTATACTAGATATGCTGATA
GTGTCAAGGGTAGATTCACAATTTCTGCTGATACATCAAAAAACACTGCT
TACTTGCAGATGAATAGCCTTAGAGCTGAGGATACAGCAGTCTACTATTG
CTCAAGATGGGGTGGGGATGGCTTCTATGCTATGGACTATTGGGGTCAAG
GAACATTGGTGACAGTATCAAGTGCTTCCACCAAGGGACCAAGCGTTTTT
CCTTTAGCCCCAAGTTCTAAGTCCACTAGTGGAGGTACCGCAGCTCTTGG
TTGTTTAGTCAAAGATTATTTCCCAGAGCCAGTTACCGTGAGTTGGAACA
GTGGTGCTTTGACTAGTGGAGTCCATACATTCCCAGCTGTTTTGCAATCT
AGTGGATTGTATTCACTCTCTAGTGTGGTTACCGTGCCAAGCTCAAGTTT
AGGAACACAAACATATATATGCAATGTGAATCATAAACCAAGCAACACTA
AAGTTGATAAGAAAGTGGAACCAAAGTCATGCGACAAAACACATACTTGC
CCTCCATGCCCTGCACCTGAATTATTGGGAGGTCCTAGTGTTTTTTTATT
TCCACCTAAACCAAAAGATACCCTTATGATTTCTAGGACACCAGAAGTTA
CTTGTGTCGTGGTCGATGTGTCCCATGAAGATCCAGAAGTTAAATTCAAT
TGGTATGTGGATGGTGTTGAAGTGCATAACGCTAAGACTAAGCCTAGGGA
GGAACAATATAATTCAACTTATAGAGTCGTTAGTGTCCTTACTGTCCTCC
ACCAAGATTGGTTGAATGGAAAGGAGTATAAATGCAAAGTCTCAAATAAG
GCTCTCCCAGCACCTATCGAAAAAACCATATCCAAGGCCAAAGGACAACC
TAGAGAGCCTCAAGTTTATACACTTCCACCTTCTAGGGAAGAAATGACAA
AGAACCAAGTGAGCCTTACATGTCTCGTTAAGGGTTTCTATCCTAGTGAC
ATTGCCGTTGAATGGGAGAGTAATGGACAACCTGAGAACAATTATAAGAC
TACACCTCCAGTCTTGGATAGTGATGGTTCTTTCTTTTTGTATTCTAAAT
TAACTGTTGACAAATCAAGATGGCAACAGGGAAATGTTTTTTCATGTTCT
GTCATGCACGAGGCTCTTCACAATCATTATACTCAAAAATCACTTAGCCT
TAGCCCAGGATAATGA
SEQ ID NO: 10
amino acid sequence of the optimized heavy
chain of trastuzumab, including signal peptide
MAKTNLFLFLIFSLLLSLSSAEVQLVESGGGLVQPGGSLRLSCAASGFNI
KDTYIHWVRQAPGKGLEWVARIYPTNGYTRYADSVKGRFTISADTSKNTA
YLQMNSLRAEDTAVYYCSRWGGDGFYAMDYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPG
SEQ ID NO: 11
nucleic acid sequence of the optimized light
chain coding sequence of trastuzumab,
including signal peptide
ATGGCTAAAACAAATCTCTTTTTATTCTTGATTTTCTCCCTTTTACTTTC
CTTAAGCTCAGCGGACATTCAAATGACTCAATCCCCATCAAGTCTTAGCG
CAAGTGTGGGAGATCGTGTCACTATTACATGTCGAGCATCTCAAGATGTG
AATACTGCTGTTGCGTGGTACCAACAAAAGCCTGGTAAAGCTCCAAAGTT
ACTTATATACAGTGCAAGCTTTCTTTATAGTGGCGTACCATCTCGATTCA
GTGGATCTCGAAGTGGAACTGACTTCACCTTGACTATCTCATCTCTACAA
CCAGAAGACTTCGCTACTTACTATTGTCAACAACATTATACAACTCCTCC
TACATTCGGGCAAGGTACCAAAGTTGAGATCAAGAGGACCGTGGCTGCAC
CAAGTGTGTTCATATTTCCTCCATCCGATGAACAATTGAAGAGTGGTACC
GCAAGCGTCGTGTGTTTATTGAATAACTTTTACCCAAGGGAAGCCAAAGT
TCAATGGAAAGTTGATAATGCTCTCCAAAGTGGAAACTCACAAGAAAGTG
TTACAGAGCAAGACTCAAAAGATTCCACTTATAGCTTATCAAGTACACTT
ACTCTCTCAAAAGCAGACTATGAAAAACACAAAGTCTACGCTTGCGAAGT
CACTCATCAAGGACTTTCTTCACCAGTTACAAAGAGTTTCAATAGAGGAG
AGTGTTAATGA
SEQ ID NO: 12
amino acid sequence of the optimized light
chain of trastuzumab, including signal
peptide
MAKTNLFLFLIFSLLLSLSSADIQMTQSPSSLSASVGDRVTITCRASQDV
NTAVAWYQQKPGKAPKLLIYSASFLYSGVPSRFSGSRSGTDFTLTISSLQ
PEDFATYYCQQHYTTPPTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

### FULL CITATIONS FOR REFERENCES REFERRED TO IN THE SPECIFICATION

Almquist, K. C., McLean, M. D., Niu, Y., Byrne, G., Olea-Popelka, F. C., Murrant, C., Barclay, J., and Hall, J. C. (2006). Expression of an anti-botulinum toxin A neutralizing single-chain Fv recombinant antibody in transgenic tobacco. Vaccine 24, 2079-2086.
Almquist K. C., Niu Y., McLean M. D., Mena F. L., Yau K. Y., Brown K., Brandle J. E. and Hall J. C. (2004) Immunomodulation confers herbicide resistance in plants. Plant Biotechnol J 2, 189-97.
Arnould, L., Gelly, M., Penault-Llorca, F., Benoit, L., Bonnetain, F., Migeon, C., Cabaret, V., Fermeaux, V., Bertheau, P., Garnier, J., et al. (2006). Trastuzumab-based treatment of HER2-positive breast cancer: an antibody-dependent cellular cytotoxicity mechanism? Br. J. Cancer 94, 259-267.
Beano, A., Signorino, E., Evangelista, A., Brusa, D., Mistrangelo, M., Polimeni, M. A., Spadi, R., Donadio, M., Ciuffreda, L., and Matera, L. (2008). Correlation between NK function and response to trastuzumab in metastatic breast cancer patients. J Transl Med 6, 25.
Ben-Kasus, T., Schechter, B., Lavi, S., Yarden, Y., and Sela, M. (2009). Persistent elimination of ErbB-2/HER2-overexpressing tumors using combinations of monoclonal antibodies: Relevance of receptor endocytosis. Proceedings of the National Academy of Sciences 106, 3294-3299.
Birch, J. R., and Racher, A. J. (2006). Antibody production. Advanced Drug Delivery Reviews 58, 671-685.
Brodsky, L. I.; Ivanov, V. V.; Kalaidzidis, Y. L.; Leontovich, A. M.; Nikolaev, V. K.; Feranchuk, S. I.; Drachev, V. A. GeneBee-NET: internet-based server for analyzing biopolymers structures. Biochemistry 1995, 60, 923-928.
Brodzik, R., Glogowska, M., Bandurska, K., Okulicz, M., Deka, D., Ko, K., van der Linden, J., Leusen, J. H. W., Pogrebnyak, N., Golovkin, M., et al. (2006). Plant-derived anti-Lewis Y mAb exhibits biological activities for efficient immunotherapy against human cancer cells. Proc. Natl. Acad. Sci. U.S.A 103, 8804-8809.
Broothaerts W, Mitchell H J, Weir B, Kaines S, Smith L M A, Yang W, Mayer J E, Roa-Rodriguez C, Jefferson R A (2005) Gene transfer to plants by diverse species of bacteria, Nature 433:629-633.
Cabanes-Macheteau, M., Fitchette-Lainé, A. C., Loutelier-Bourhis, C., Lange, C., Vine, N. D., Ma, J. K., Lerouge, P., and Faye, L. (1999). N-Glycosylation of a mouse IgG expressed in transgenic tobacco plants. Glycobiology 9, 365-372.
Chadd, H. E., and Chamow, S. M. (2001). Therapeutic antibody expression technology. Current Opinion in Biotechnology 12, 188-194.
Chames, P., Van Regenmortel, M., Weiss, E., and Baty, D. (2009). Therapeutic antibodies: successes, limitations and hopes for the future. Br. J. Pharmacol 157, 220-233.
Chung S. M., Vaidya M. and Tzfira T. (2006) Agrobacterium is not alone: gene transfer to plants by viruses and other bacteria. Trends Plant Sci 11, 1-4.
CNR National Research Council. SpliceView. WebGene Webpage available on-line at www.itb.cnr.it/sun/webgene/, 2005.
Cobleigh, M. A., Vogel, C. L., Tripathy, D., Robert, N. J., Scholl, S., Fehrenbacher, L., Wolter, J. M., Paton, V., Shak, S., Lieberman, G., et al. (1999). Multinational Study of the Efficacy and Safety of Humanized Anti-HER2 Monoclonal Antibody in Women Who Have HER2-Overexpressing Metastatic Breast Cancer That Has Progressed After Chemotherapy for Metastatic Disease. J Clin Oncol 17, 2639.
Colgan R., Atkinson C. J., Paul M., Hassan S., Drake P. M., Sexton A. L., Santa-Cruz S., James D., Hamp K., Gutteridge C. and Ma J. K. Optimisation of contained Nicotiana tabacum cultivation for the production of recombinant protein pharmaceuticals. Transgenic Res 19, 241-56.
Conley, A. J. 2009 Chapter 4: Recombinant Protein Production in a variety of Nicotiana hosts: A comparative analysis. In: Approaches for Enhancing Recombinant Protein Proeuction in Transgenic Plants; PhD Thesis, The School of Graduate and Postdoctoral Studies, The University of Western Ontario, London, Ontario, Canada.
Dimitrov, D. S., and Marks, J. D. (2009). Therapeutic Antibodies: Current State and Future Trends—Is a Paradigm Change Coming Soon? In Therapeutic Antibodies, pp. 1-27. Available at: http://dx.doi.org/10.1007/978-1-59745-554-1_{—}1 [Accessed Sep. 30, 2009].
Entelchon GmbH. Backtranslation tool. Available on-line at www.entelechon.com/index.php?id) tools/backtranslation & lang)eng, 2005.
Fischer, R., Schillberg, S., and Twyman, R. M. (2009). Molecular Farming of Antibodies in Plants. In Recent Advances in Plant Biotechnology, pp. 35-63. Available at: http://dx.doi.org/10.1007/978-1-4419-0194-1_{—}3 [Accessed Dec. 17, 2009].
Floss, D. M., Sack, M., Stadlmann, J., Rademacher, T., Scheller, J., Stöger, E., Fischer, R., and Conrad, U. (2008). Biochemical and functional characterization of anti-HIV antibody-ELP fusion proteins from transgenic plants. Plant Biotechnology Journal 6, 379-391.
Gardiner-Garden, M.; Frommer, M. CpG islands in vertebrate genomes. J. Mol. Biol. 1987, 196, 261-282.
Gelvin S. B. (2003) Agrobacterium-mediated plant transformation: the biology behind the “gene-jockeying” tool. Microbiol Mol Biol Rev 67, 16-37.
GeneBee Molecular Biology Server. RNA secondary structure prediction.

Available on-line at www.genebee.msu.su/services/rna2_reduced.html; 2001.
Gilbert, N. (2009). Fees delay pharmed drug. Nature 458, 951.
Giritch A., Marillonnet S., Engler C., van Eldik G., Botterman J., Klimyuk V. and Gleba Y. (2006) Rapid high-yield expression of full-size IgG antibodies in plants coinfected with noncompeting viral vectors. Proc Natl Acad Sci USA 103, 14701-6.
Gomord, V., Sourrouille, C., Fitchette, A., Bardor, M., Pagny, S., Lerouge, P., and Faye, L. (2004). Production and glycosylation of plant-made pharmaceuticals: the antibodies as a challenge. Plant Biotechnol. J 2, 83-100.
Hiatt, A., Caffferkey, R., and Bowdish, K. (1989). Production of antibodies in transgenic plants. Nature 342, 76-78.
Horsch R. B., Fry J. E., Hoffmann N. L., Eichholtz D., Rogers S. G. and Fraley R. T. 1985. A simple and general method for transferring genes into plants. Science 227: 1229-1231.
Hynes, N. E., and Stern, D. F. (1994). The biology of erbB-2/neu/HER-2 and its role in cancer. Biochim. Biophys. Acta 1198, 165-184.
Ilham A. Shahmuradov, Alex J. Gammerman, John M. Hancock, Peter M. Bramley and Victor V. Solovyev. 2003. PlantProm: a database of plant promoter sequences. Nucleic Acids Research, 2003, Vol. 31, No. 1 114-117.
Jamal, A., Ahn, M., Song, M., Oh, E., Hong, J., Choo, Y., Ko, K., Han, Y. S., Oh, S. H., Van Der Linden, J., et al. (2009). Biological Validation of Plant-Derived Anti-Human Colorectal Cancer Monoclonal Antibody CO17-1A. Hybridoma (Larchmt). Available at: http://www.ncbi.nlm.nih.gov/pubmed/19196053 [Accessed Dec. 17, 2009].
Karg, S. R., and Kallio, P. T. (2009). The production of biopharmaceuticals in plant systems. Biotechnol. Adv. Available at: http://www.ncbi.nlm.nih.gov/pubmed/19647060 [Accessed Sep. 30, 2009].
Ko, K., Brodzik, R., and Steplewski, Z. (2009). Production of Antibodies in Plants: Approaches and Perspectives. In Plant-produced Microbial Vaccines, pp. 55-78. Available at: http://dx.doi.org/10.1007/978-3-540-70868-1_{—}4 [Accessed Sep. 30, 2009].
Ko, K., Steplewski, Z., Glogowska, M., and Koprowski, H. (2005). Inhibition of tumor growth by plant-derived mAb. Proc Natl Acad Sci USA. 102, 7026-7030.
Ko, K., Tekoah, Y., Rudd, P. M., Harvey, D. J., Dwek, R. A., Spitsin, S., Hanlon, C. A., Rupprecht, C., Dietzschold, B., Golovkin, M., et al. (2003). Function and glycosylation of plant-derived antiviral monoclonal antibody. Proc Natl Acad Sci USA. 100, 8013-8018.
Kozak M. (1984) Compilation and analysis of sequences upstream from the translational start site in eukaryotic mRNAs. Nucleic Acids Res 12, 857-72.
Lan-Ying Lee, Maria E. Kononov, Burgund Bassuner, Bronwyn R. Frame, Kan Wang, and Stanton B. Gelvin 2007 Novel Plant Transformation Vectors Containing the Superpromoter Plant Phys 145: 1294-1300.
Le, X., Claret, F., Lammayot, A., Tian, L., Deshpande, D., LaPushin, R., Tari, A. M., and Bast, R. C. (2003). The role of cyclin-dependent kinase inhibitor p27Kip1 in anti-HER2 antibody-induced G1 cell cycle arrest and tumor growth inhibition. J. Biol. Chem 278, 23441-23450.
Leong, S. S., and Chen, W. N. (2008). Preparing recombinant single chain antibodies. Chemical Engineering Science 63, 1401-1414.
Li, Q.; Hunt, A. A near-stream element in a plant polyadenylation signal consist of more than six nucleotides. Plant Mol. Biol. 1995, 28, 927-934.
Ma, J. K., Lehner, Thomas, Stabila, P., Fux, C. I., and Hiatt, A. (1994). Assembly of monoclonal antibodies with IgG1 and IgA heavy chain domains in transgenic tobacco plants. European Journal of Immunology 24, 138.
Ma, J. K., Hiatt, A., Hein, M., Vine, N. D., Wang, F., Stabila, P., Dolleweerd, C. V., Mostov, K., and Lehner, T. (1995). Generation and Assembly of Secretory Antibodies in Plants. Science 268, 716-719.
Makvandi-Nejad S., McLean M. D., Hirama T., Almquist K. C., Mackenzie C. R. and Hall J. C. (2005) Transgenic tobacco plants expressing a dimeric single-chain variable fragment (scfv) antibody against Salmonella enterica serotype Paratyphi B. Transgenic Res 14, 785-92.
Marillonnet, S., Thoeringer, C., Kandzia, R., Klimyuk, V., and Gleba, Y. (2005). Systemic Agrobacterium tumefaciens-mediated transfection of viral replicons for efficient transient expression in plants. Nat. Biotechnol 23, 718-723.
McLean, M. D., Almquist, K. C., Niu, Y., Kimmel, R., Lai, Z., Schreiber, J. R., and Hall, J. C. (2007). A Human Anti-Pseudomonas aeruginosa Serotype O6ad Immunoglobulin G1 Expressed in Transgenic Tobacco Is Capable of Recruiting Immune System Effector Function In Vitro. Antimicrob. Agents Chemother. 51, 3322-3328.
Molina, M. A., Codony-Servat, J., Albanell, J., Rojo, F., Arribas, J., and Baselga, J. (2001). Trastuzumab (herceptin), a humanized anti-Her2 receptor monoclonal antibody, inhibits basal and activated Her2 ectodomain cleavage in breast cancer cells. Cancer Res 61, 4744-4749.
Mori, K., Iida, S., Yamane-Ohnuki, N., Kanda, Y., Kuni-Kamochi, R., Nakano, R., Imai-Nishiya, H., Okazaki, A., Shinkawa, T., Natsume, A., et al. (2007). Non-fucosylated therapeutic antibodies: the next generation of therapeutic antibodies. Cytotechnology 55, 109-114.
Nakamura, Y. Codon usage database. Available on-line at www.kazusa.or.jp/codon; 2005.
Ni M, Cui D, Einstein J, Narasimhulu S, Vergara C E, Gelvin S B (1995) Strength and tissue specificity of chimeric promoters derived from the octopine and mannopine synthase genes. Plant J 7: 661-676.
Nissim, A., and Chernajovsky, Y. (2008). Historical development of monoclonal antibody therapeutics. Handb Exp Pharmacol, 3-18.
Olea-Popelka, F., McLean, M. D., Horsman, J., Almquist, K., Brandle, J. E., and Hall, J. C. (2005). Increasing expression of an anti-picloram single-chain variable fragment (ScFv) antibody and resistance to picloram in transgenic tobacco (Nicotiana tabacum). J. Agric. Food Chem 53, 6683-6690.
Ohme-Takagi, M.; Taylor, C.; Newman, T.; Green, P. The effect of sequences with high AU content on mRNA stability in tobacco. Proc. Natl. Acad. Sci. U.S.A. 1993, 90, 11811-11815.
Ramessar, K., Rademacher, T., Sack, M., Stadlmann, J., Platis, D., Stiegler, G., Labrou, N., Altmann, F., Ma, J., Stöger, E., et al. (2008). Cost-effective production of a vaginal protein microbicide to prevent HIV transmission. Proc. Natl. Acad. Sci. U.S.A 105, 3727-3732.
Rodríguez, M., Ramirez, N. I., Ayala, M., Freyre, F., Perez, L., Triguero, A., Mateo, C., Selman-Housein, G., Gavilondo, J. V., and Pujol, M. (2005). Transient expression in tobacco leaves of an aglycosylated recombinant antibody against the epidermal growth factor receptor. Biotechnology and Bioengineering 89, 188-194.
Roque, A. C., Silva, C. S., and Taipa, M. Â. (2007). Affinity-based methodologies and ligands for antibody purification: Advances and perspectives. Journal of Chromatography A 1160, 44-55.
Rothnie, H. M. Plant mRNA 3-end formation. Plant Mol. Biol. 1996, 32, 43-61.
Sack, M., Paetz, A., Kunert, R., Bomble, M., Hesse, F., Stiegler, G., Fischer, R., Katinger, H., Stoeger, E., and Rademacher, T. (2007). Functional analysis of the broadly neutralizing human anti-HIV-1 antibody 2F5 produced in transgenic BY-2 suspension cultures. FASEB J. 21, 1655-1664.
Samac, D. A., Hironaka, C. M., Yallaly, P. E., and Shah, D. M. (1990). Isolation and Characterization of the Genes Encoding Basic and Acidic Chitinase in Arabidopsis thaliana. Plant Physiol 93, 907-914.
Shapiro, M. B.; Senapathy, P. RNA splice junctions of different classes of eukaryotes: sequence statistics and functional implications in gene expression. Nucleic Acids Res. 1987, 5, 7155-7174.
Slamon, D. J., Clark, G. M., Wong, S. G., Levin, W. J., Ullrich, A., and McGuire, W. L. (1987). Human breast cancer: correlation of relapse and survival with amplification of the HER-2/neu oncogene. Science 235, 177-182.
Slamon, D. J., Godolphin, W., Jones, L. A., Holt, J. A., Wong, S. G., Keith, D. E., Levin, W. J., Stuart, S. G., Udove, J., and Ullrich, A. (1989). Studies of the HER-2/neu proto-oncogene in human breast and ovarian cancer. Science 244, 707-712.
Stoger, E., Sack, M., Nicholson, L., Fischer, R., and Christou, P. (2005). Recent progress in plantibody technology. Curr. Pharm. Des 11, 2439-2457.
Strasser, R., Castilho, A., Stadlmann, J., Kunert, R., Quendler, H., Gattinger, P., Jez, J., Rademacher, T., Altmann, F., Mach, L., et al. (2009). Improved virus neutralization by plant-produced anti-HIV antibodies with a homogeneous beta1,4-galactosylated N-glycan profile. J. Biol. Chem 284, 20479-20485.
Strasser, R., Stadlmann, J., Schähs, M., Stiegler, G., Quendler, H., Mach, L., Glössl, J., Weterings, K., Pabst, M., and Steinkellner, H. (2008). Generation of glyco-engineered Nicotiana benthamiana for the production of monoclonal antibodies with a homogeneous human-like N-glycan structure. Plant Biotechnol. J 6, 392-402.
Suzuki, E., Niwa, R., Saji, S., Muta, M., Hirose, M., Iida, S., Shiotsu, Y., Satoh, M., Shitara, K., Kondo, M., et al. (2007). A nonfucosylated anti-HER2 antibody augments antibody-dependent cellular cytotoxicity in breast cancer patients. Clin. Cancer Res 13, 1875-1882.
Vaquero, C., Sack, M., Chandler, J., Drossard, J., Schuster, F., Monecke, M., Schillberg, S., and Fischer, R. (1999). Transient expression of a tumor-specific single-chain fragment and a chimeric antibody in tobacco leaves. Proceedings of the National Academy of Sciences of the United States of America 96, 11128-11133.
Vézina, L., Faye, L., Lerouge, P., D'Aoust, M., Marquet-Blouin, E., Burel, C., Lavoie, P., Bardor, M., and Gomord, V. (2009). Transient co-expression for fast and high-yield production of antibodies with human-like N-glycans in plants. Plant Biotechnol. J 7, 442-455.
Yu D., McLean M. D., Hall J. C. and Ghosh R. (2008) Purification of a human immunoglobulin G1 monoclonal antibody from transgenic tobacco using membrane chromatographic processes. J Chromatogr A 1187, 128-37.
Zeitlin, L., Olmsted, S. S., Moench, T. R., Co, M. S., Martinell, B. J., Paradkar, V. M., Russell, D. R., Queen, C., Cone, R. A., and Whaley, K. J. (1998). A humanized monoclonal antibody produced in transgenic plants for immunoprotection of the vagina against genital herpes. Nat Biotech 16, 1361-1364.
Zhou, B. P., Liao, Y., Xia, W., Spohn, B., Lee, M. H., and Hung, M. C. (2001). Cytoplasmic localization of p21Cip1/WAF1 by Akt-induced phosphorylation in HER-2/neu-overexpressing cells. Nat. Cell Biol 3, 245-252.

1. A method of making an antibody or fragment thereof that binds to a human epidermal growth factor receptor (HER) in a plant comprising:
(a) introducing a nucleic acid molecule encoding a heavy chain variable region and a nucleic acid encoding a light chain variable region of the antibody into a plant or plant cell; and
(b) growing the plant or plant cell to obtain a plant that expresses the antibody or antibody fragment.

2. A method according to claim 1 wherein the nucleic acid molecule encoding a heavy chain variable region and the nucleic acid molecule encoding a light chain variable region are introduced on separate vectors.

3. A method according to claim 1 wherein the nucleic acid sequence encodes the heavy chain variable region shown in SEQ ID NO:2.

4. A method according to claim 3 wherein the nucleic acid sequence encoding the heavy chain variable region has a sequence shown in SEQ ID NO:1.

5. A method according to claim 1 wherein the nucleic acid sequence encodes the light chain variable region shown in SEQ ID NO:4.

6. A method according to claim 5 wherein the nucleic acid sequence encoding the light chain variable region has a sequence shown in SEQ ID NO:3.

7. A method according to claim 1 wherein a nucleic acid molecule encoding a heavy chain and a nucleic acid molecule encoding a light chain is introduced in step (a).

8. A method according to claim 7 wherein the nucleic acid sequence encodes the heavy chain shown in SEQ ID NO:6.

9. A method according to claim 8 wherein the nucleic acid sequence encoding the heavy chain has the sequence shown in SEQ ID NO:5.

10. A method according to claim 7 wherein the nucleic acid sequence encodes the light chain shown in SEQ ID NO:8.

11. A method according to claim 10 wherein the nucleic acid sequence encoding the light chain has the sequence shown in SEQ ID NO:7.

12. A method according to claim 7 wherein the nucleic acid sequence encodes the heavy chain shown in SEQ ID NO:10.

13. A method according to claim 12 wherein the nucleic acid sequence encoding the heavy chain has the sequence shown in SEQ ID NO:9.

14. A method according to claim 7 wherein the nucleic acid sequence encodes the light chain shown in SEQ ID NO:12.

15. A method according to claim 14 wherein the nucleic acid sequence encoding the light chain has the sequence shown in SEQ ID NO:11.

16. A method according to claim 1 wherein the plant is a tobacco plant.

17. An antibody or antibody fragment prepared according to a method of claim 1.

18. A transgenic plant that expresses an antibody that binds to a human epidermal growth factor receptor (HER) comprising a nucleic acid molecule encoding a heavy chain variable region and a nucleic acid encoding a light chain variable region of the antibody.

19. The transgenic plant according to claim 18 comprising a nucleic acid sequence encoding the heavy chain of SEQ ID NO:9 and a nucleic acid sequence encoding the light chain of SEQ ID NO:11.

20. A method of treating a cancer that expresses HER comprising administering an effective amount of an antibody according to claim 17 to a subject in need thereof.

21. A method according to claim 17 wherein the cancer is breast cancer.
